(19) United States
(12) Patent Application Publication
(10) Pub. No.: US 2020/0248138 A1
(43) Pub. Date: Aug. 6, 2020

(54) METHOD FOR INDUCING DIFFERENTIATION OF PLURIPOTENT STEM CELLS INTO GERMLINE STEM CELL-LIKE CELLS

(71) Applicant: Kyoto University, Kyoto (JP)

(72) Inventors: Mitinori SAITOU, Kyoto (JP); Yukiko ISHIKURA, Kyoto (JP)

(73) Assignee: Kyoto University, Kyoto (JP)

(21) Appl. No.: 16/619,361

(22) PCT Filed: Jun. 6, 2018
(86) PCT No.: PCT/JP2018/021775

§ 371 Date: Apr. 27, 2020

(30) Foreign Application Priority Data
Jun. 7, 2017 (JP) 2017-113054

(51) Int. Cl. C12N 5/0735; C12N 5/074; A01K 67/027; A61D 19/00

(52) U.S. Cl. CPC C12N 5/0611; C12N 5/0607; A01K 2227/10; A61D 19/00; A01K 67/027

(57) ABSTRACT

Provided are a method for producing a spermatogenic stem cell-like cell from a primordial germ cell-like cell derived from an isolated pluripotent stem cell in vitro, the method including
(1) a step of coculturing a primordial germ cell-like cell with a gonad somatic cell in suspension to give reconstituted testis, and
(2) a step of culturing the obtained reconstituted testis at gas/liquid interface to induce a DDX4-positive and PLZF-positive cell in the reconstituted testis; and
   a method for producing a GSC-like cell, including dissociating a spermatogenic stem cell-like cell obtained by the method from the reconstituted testis, and culturing the cell under conditions that can induce a germline stem cell from the spermatogenic stem cell.

### TECHNICAL FIELD

The present invention relates to a method for inducing a germline stem cell-like cell from a pluripotent stem cell via an epiblast-like cell, a primordial germ cell-like cell in vitro, and a method for inducing a normal spermatozoon in the testis of adult animal from the germline stem cell-like cell.

### BACKGROUND ART

The main problem in the developmental biology is reconstitution of an essential developmental pathway in vitro, and this not only provides an opportunity for a new experiment but also is useful as a basis of medical application. In multicellular organisms, the germline cells have been conferred with an essential function of ensuring the creation of new organisms, whereby genetic information and epigenetic information are inherited across generations. Thus, reconstituting the development of germline cells in vitro is generally of intrinsic significance in life science. Some attempts have been made to generate a gamete or a progenitor cell thereof (primordial germ cell; sometimes to be abbreviated as “PGC”) in vitro from embryonic stem cells (sometimes to be abbreviated as “ES cell” and “ESC”) derived from an inner cell mass of mouse and human blastocysts. However, all of these attempts involved random differentiation of ESC as embryoid under chemically undefined conditions, and were dependent on the spontaneous expression of one or more kinds of marker genes. As a result, these attempts were inefficient in obtaining the cells of interest. Furthermore, it has not been demonstrated that the generated cells contribute to the creation of a healthy offspring.

The present inventors previously established a culture system for inducing ES cell/induced pluripotent stem cell (sometimes to be abbreviated as “iPS cell”, “iPSC”) into epiblast-like cell (sometimes to be abbreviated as “EpiLC”) by using a cytokine including activin A and basic fibroblast growth factor (bFGF), and thereafter inducing same into primordial germ cell-like cell (sometimes to be abbreviated as “PGC-like cell”, “PGCLC”) by using a cytokine including BMP4 (patent document 1, non-patent documents 1 and 2). Furthermore, they transplanted the PGCLC into the testis or under the egg sac of a neonatal mouse, differentiated same into spermatozoon or ovum, and successfully obtained a normal offspring therefrom (patent document 1, non-patent documents 1 and 2). In addition, they also succeeded in inducing ovum from pluripotent stem cell (sometimes to be abbreviated as “PSC”) via PGCLC in vitro.

As for male, one of the goals has been to induce a spermatogenic stem cell, which is the pre-spermatozoon cell, from a pluripotent stem cell via PGC. Spermatogenic stem cell is a cell that produces spermatozoon for entire lifetime, is rarely found in the testis of adults, and is said to be the only stem cell in the germline. Methods for establishing a long-term culture strain of spermatogenic stem cell (germ line stem cell; GSC) have been studied, but the mechanism of inducing differentiation of spermatogenic stem cell from PGC includes many unknown aspects, and establishment of a culture system for inducing GSC-like cell from pluripotent stem cell via PGCLC in vitro has been desired.

### DOCUMENT LIST

### Patent Document

patent document 1: WO 2012/020687

### Non-Patent Documents

non-patent document 1: Hayashi, K., Ohta, H., Kurimoto, K., Aramaki, S. & Saitou, M. Reconstitution of the mouse germ cell specification pathway in culture by pluripotent stem cells. Cell 146, 519-532 (2011).
non-patent document 2: Hayashi, K. et al. Offspring from oocytes derived from in vitro primordial germ cell-like cells in mice. Science 338, 971-975 (2012).

### SUMMARY OF THE INVENTION

### Problems to be Solved by the Invention

Therefore, the present invention aims to provide a method for inducing a GSC-like cell from pluripotent stem cell-derived PGCLC in vitro, and to provide a method for differentiating the GSC-like cell into spermatozoon and efficiently producing an offspring to which PSC contributes.

### Means of Solving the Problems

In mouse, by 12.5 days of gestational age (E12.5), PGC is surrounded by gonad somatic cells that are the source of testes in the future, called pro-spermatogonium, and thereafter differentiates into spermatogonial and spermatogenic stem cell at around 5 days of age. Thus, the present inventors focused on the cell environment at the time point when PGC becomes pro-spermatogonium, cultured PGCLC derived from mouse ESC in suspension together with gonad somatic cell of mouse embryo (E12.5) to allow for aggregation to produce “reconstituted testis”, and examined the culture conditions for inducing differentiation into spermatogenic stem cell. As a result, they could determine the culture conditions under which PGCLC differentiates into a cell exhibiting properties equivalent to those of spermatogenic stem cell. When the cell was further cultured, it proliferated in the same manner as GS cell derived from a living body, and it was confirmed that long-term culture of 4 months or more was possible. The present inventors named this cell line a germline stem cell-like cell (GSCLC). The present inventors further transplanted GSCLC into the testes of neonate (7 days of age) and adult (8 weeks of age) germ cell-deficient mice. As a result, a part thereof differentiated into spermatozoon, and it was confirmed that a healthy offspring could be obtained by microinsemination with the obtained spermatozoon with an oocyte, which resulted in the completion of the present invention.

That is, the present invention relates to the following:

[1] A method for producing a spermatogenic stem cell-like cell from a primordial germ cell-like cell (PGCLC) derived from an isolated pluripotent stem cell (PSC) in vitro, the method comprising
(1) a step of coculturing PGCLC with a gonad somatic cell in suspension to give reconstituted testis, and
(2) a step of culturing the obtained reconstituted testis at gas/liquid interface to induce a DDX4-positive and PLZF-positive cell in the reconstituted testis.
[2] A method for producing a GSC-like cell (GSCLC), comprising dissociating a spermatogenic stem cell-like cell obtained by the method of [1] from the reconstituted testis, and culturing the cell under conditions that can induce a germline stem cell (GSC) from the spermatogenic stem cell.
[3] An isolated GSCLC having the following properties:
(a) derived from isolated PSC,
(b) having expression levels equivalent to those of GSC as regards
(i) a gene selected from the group consisting of Ddx4, Daz1, Gfra1, Ret, Piwil2, Itga6, Kit, Plzf, Piwil4 and Id4,
(ii) a surface marker selected from the group consisting of CD9, SSEA1, INTEGRINβ1, INTEGRINα6, KIT and GFRα1, and
(iii) a transcription factor selected from the group consisting of PLZF and ID4,
(c) can be maintained or expanded at a proliferation rate equivalent to that of GSC,
(d) when GSCLC is transplanted into an adult testis,
(i) a proportion of seminiferous tubule having GFRα1-positive cells to seminiferous tubule with colonized transplanted cells is equivalent to that when GSC is transplanted, and
(ii) a proportion of seminiferous tubule having SCP3-positive cells to seminiferous tubule with colonized transplanted cells is lower than that when GSC is transplanted,
(e) microinsemination with a sperm cell obtained by transplantation of the GSCLC into an adult testis produces normal offspring.
[4] A method for producing a fertile sperm cell comprising transplanting GSCLC obtained by the method of [2] or GSCLC of [3] into the testis of a mammal.
[5] A method for producing an offspring with contribution of isolated PSC to the whole body, comprising fertilizing an oocyte by a sperm cell obtained by the method of [4].

### Effect of the Invention

According to the present invention, two problems of the conventional PGCLC, namely, (1) long-term maintenance culture cannot be performed and (2) differentiation into spermatozoon occurs only in neonatal testis, can be solved.

### BRIEF DESCRIPTION OF THE DRAWINGS

FIG. 1 shows the development of reconstituted testis in vitro. (A) Scheme for production and culture of reconstituted testis under two conditions. (B) Representative images of development of reconstituted testes for 3 weeks (Condition 2). Bright Field images (BF), AAG fluorescence, and merge thereof are shown. bar: 200 μm. (C) Expression of GATA4 and SOX9 (left) or DDX4 and PLZF (right) in reconstituted testis at d14 and d21 (condition 2) or testis at E13.5 and P3. bar: 50 μm. Cells derived from PGCLC and endogenous germ cells are respectively identified by GFP and DDX4 (second row). DAPI staining and merge are also shown. (D) Proportion of differentiation of d4 PGCLC or E12.5 PGC into DDX4(+)/PLZF(−) and DDX4(+)/PLZF(+) cells in reconstituted testis. (E) Long-term culture of reconstituted testis (Condition 1). (left) Morphology of reconstituted testis cultured for 28 days. bar: 200 μm. (right) Expression of DDX4 (ii), SCP3 (iii), and GFP (iv) in sections of reconstituted testis cultured for 30 days. (i) is DAPI. bar: 10 μm. See also FIG. 2.

FIG. 2 shows reconstituted testis in vitro and relates to FIG. 1. (A) Representative images of developments of reconstituted testis for 3 weeks under Condition 1. Bright Field images (BF, top), GFP fluorescence by AAG (middle), and merge thereof (bottom). bar: 200 μm. (B) IF analysis of expression of GATA4 and SOX9 (left) or DDX4 and PLZF (right) in reconstituted testis at d14 and d21 (Condition 1), or reconstituted testis at E13.5 and days after birth (P)3. bar: 50 μm. PGCLC-derived cell and endogenous germ cell were respectively identified by GFP and DDX4 (second row). DAPI staining and merge are respectively shown in upper and lower rows. (C) IF analysis of expression of DDX4 and PLZF in reconstituted testis at d21 (Conditions 1 (left) and 2 (right)). Endogenous germ cell was identified as GFP(−)/DDX4(+) cell. DAPI staining and merge are respectively shown in upper and lower rows. bar: 50 μm. (D) Long-term culture of reconstituted testis having AAG(+)PGC in vivo (Condition 1). (left) Morphology of reconstituted testis cultured for 49 days. Bright Field image (BF), GFP fluorescence by AAG, and merge thereof (bottom) are shown. bar: 200 μm. (right) IF analysis of expression of DDX4 (ii), SCP3 (iii), and GFP (iv) in sections of reconstituted testis cultured for 54 days is shown together with (i) DAPI. bar: 10 μm.

FIG. 3 shows induction of germline stem cell-like cell from reconstituted testis. (A) Scheme of induction of GSCLC from reconstituted testis. (B) (top) Representative images of colony of AAG(+) cells during induction of GSCLC. BF image, AAG fluorescence, and merge thereof are shown. (bottom) Images of GSCLC cell line (GSCLC1) at 9 passages. bar: 100 μm. (C) Proliferation of GSCLC (GSCLCs 1, 2) and GSC (GSC1). 2×10⁵ GSCLC/GSC were seeded and proliferation thereof was evaluated every 6 days or 7 days. (D) Gene expression levels in GSCLC (GSCLCs 1, 4) and GSC (GSCs 1, 2) as measured by qPCR. DCt (difference in threshold value cycle [Ct] value) from average Ct value (set to 0) of two housekeeping genes Arbp and Ppia was plotted for each gene. Red dotted line shows Ct value±1 of average Cts of GSC. (E) Expression levels (red plot) of surface markers in GSCLC (GSCLCs 1, 4) and GSC (GSC1) as measured by FACS analysis. Blue plot shows fluorescence intensity by an isotype-matched control antibody. (F) Expression (shown with DAPI) of GFP, ID4, and PLZF in colonies of GSCLC (GSCLC1) and GSC (GSC1) and merge thereof. bar: 50 μm. See also FIG. 4.

FIG. 4 shows a germline stem cell-like cell (GSCLC) derived from reconstituted testis and relates to FIG. 3. (A) Karyotype analysis of GSC1, GSCLC1, and GSCLC4. (B) (left) Scheme for directly culturing d4 PGCLC under GSC induction conditions. (middle) Images of Bright Field (top) or GFP (AAG) fluorescence (bottom) d4 PGCLC culture under GSC induction conditions for 1, 2 and 4 days. bar: 100 μm. Image of representative alkaline phosphatase (AP) staining in 24 well is shown on the right. bar: 1 mm. (right) Efficiency of inducing AP positive colony from d4 or d6 PGCLC cultured under GSC induction conditions. Mean is shown with a bar. 1,000-3,000 PGCLCs were seeded in each well and the number of AP positive colonies at d7 was counted.

FIG. 5 shows formation of GSCLC-derived spermatozoon and fertile offspring. Representative BF and AAG-fluorescence images of testis (A) or isolated seminiferous tubule (B) of W/W^{v} mouse at 10 weeks after transplantation of (A and B) GSC (GSC1), d4 PGCLC (A alone) and GSCLC (GSCLCs 1, 4). The enclosed regions were enlarged and it was clarified that AAG(+) cells were colonized only in the basal region of seminiferous tubule. Dot fluorescence (second from the left, bottom) found in d4 PGCLC transplantation is autofluorescence. Histological sections stained with H&E are shown in (B). bar of (A); 1 mm, bar of (B): 100 μm (left), 50 μm (right). (C) Expression (shown with DAPI) of GFP, GFRα1 and PLZF (left) or GFP, SCP and PLZF (right) in seminiferous tubule sections of W/W^{v} mouse on week 10 post-transplantation of GSCLC (GSCLC1) or GSC (GSC1). bar: 50 μm. (bottom) Representative proportion of seminiferous tubule having GFRα1(+) (left) or SCP3(+) (right) cells between tubules colonized by transplantation of GSC (GSC1) or GSCLC (GSCLCs 1, 4). (D) Images of BF (i, iii, v) and AAG-fluorescence (ii, iv, vi) of spermatozoa (I, ii, v, vi) and spermatids (iii, iv) derived from GSCLC1 (i-iv) or GSCLC3 (v, vi). bar of (ii, vi): 10 μm. bar of (iv): 50 μm. (E) Zygote in pronuclear stage (i), 2-cell embryo (ii), offspring (iii, iv), and placenta (iii) produced by ICSI of GSCLC-derived spermatozoon. (F) Fertile male offspring (agouti) and derived from spermatozoon derived from GSCLC1. (G) Development of oocyte injected with spermatozoon derived from GSCLC and GSC. See also FIG. 6.

FIG. 6 shows GSCLC-derived spermatozoon formation and offspring and relates to FIG. 5. (A) Transplantation of 15 strains of GSCLC derived from reconstituted testis under Conditions 1 and 2 into testis of a living body and spermatogenesis. (B) Number of colonies accompanied by or unaccompanied by spermatogenesis in testes transplanted with GSCLCs 1, 2 and 3. (C, D) Body weight (C) and placenta weight (D) of offspring derived from GSCLC1 and GSC1. The p value was obtained by Student's t-test. (E) Combined bisulfite restriction analysis (COBRA) of imprint of offspring derived from spermatozoon derived from GSCLCs 1 and 3. As previously reported (Lee et al., 2009), the methylation state of H19, Peg10, Igf2r, Meg3IG, and main CpG of Snrpn was analyzed. U: not digested. D: digested with respective enzymes. Length of undigested (black triangle) and digested (white triangle) fragments. All offsprings derived from spermatozoa derived from GSCLCs 1 and 3 showed clearly normal imprinting patterns.

FIG. 7 shows a culture scheme for obtaining reconstituted testis containing a spermatogenic stem cell-like cell from cultured PGCLC in Example 2.

FIG. 8 shows time-course changes in the expression of various PGC markers in the reconstituted testis cultured by gas/liquid interfacial culture.

FIG. 9 shows establishment of GSCLC from reconstituted testis on day 14 of gas/liquid interfacial culture.

### DESCRIPTION OF EMBODIMENTS

[I] Method for Producing Spermatogenic Stem Cell-Like Cell from Primordial Germ Cell-Like Cell (PGCLC)

The present invention provides a method for producing a spermatogenic stem cell-like cell from PGCLC derived from an isolated pluripotent stem cell in vitro. The method includes

(1) a step of coculturing PGCLC with a gonad somatic cell in suspension to give reconstituted testis, and
(2) a step of culturing the obtained reconstituted testis at gas/liquid interface to induce a DDX4-positive and PLZF-positive cell in the reconstituted testis.
1. Production Primordial Germ Cell-Like Cell (PGCLC) from Pluripotent Stem Cell

PGCLC used in the present invention may be any as long as, it is induced from an isolated pluripotent stem cell in vitro and has properties equivalent to those of PGC. For example, PGCLCs described in the aforementioned patent document 1 and non-patent document 1 can be mentioned. The PGCLC can be produced from isolated PSC by the method shown below via an epiblast-like cell (EpiLC).

The pluripotent stem cell to be used as a starting material for PGCLC production may be any isolated undifferentiated cell as long as it has “self-renewal potential” permitting proliferation while maintaining an undifferentiated state, and “differentiation pluripotency” permitting differentiation into all three primary germ layers. Being “isolated” here means being placed in the state of from in vivo to in vitro and it does not necessarily require purification. As the isolated pluripotent stem cell, for example, iPS cell, ES cell, embryonic germ (EG) cell, embryonic carcinoma (EC) cell and the like can be mentioned, with preference given to iPS cell or ES cell.

The method of the present invention is applicable to any mammalian species for which any PSC has been established or can be established. As the mammal, for example, human, mouse, rat, monkey, dog, swine, bovine, cat, goat, sheep, rabbit, guinea pig, hamster and the like can be mentioned. It is preferably human, mouse, rat, monkey, dog or the like, more preferably human or mouse.

### (1) Production of Pluripotent Stem Cell

### (i) ES Cell

Pluripotent stem cell can be obtained by a method known per se. For example, as a production method of ES cells, a method including culturing inner cell mass of mammalian blastocyst stage (see e.g., Manipulating the Mouse Embryo: A Laboratory Manual, Second Edition, Cold Spring Harbor Laboratory Press (1994)), a method for culturing early embryo produced by somatic cell nuclear transplantation (Wilmut et al., Nature, 385, 810(1997); Cibelli et al., Science, 280, 1256(1998); Akira Iritani et al., protein nucleic acid enzyme, 44, 892(1999); Baguisi et al., Nature Biotechnology, 17, 456(1999); Wakayama et al., Nature, 394, 369(1998); Wakayama et al., Nature Genetics, 22, 127(1999); Wakayama et al., Proc. Natl. Acad. Sci. USA, 96, 14984(1999); Rideoutlll et al., Nature Genetics, 24, 109(2000)) and the like can be mentioned, but the method is not limited thereto. ES cell can be obtained from given institutions and a commercially available product can also be purchased. For example, human ES cell lines H1 and H9 are available from WiCell Institute of University of Wisconsin, and KhES-1, KhES-2 and KhES-3 are available from Institute for Frontier Medical Science, Kyoto University. When ES cell is produced by somatic cell nuclear transplantation, the kind of somatic cells and the source of somatic cell collection are the same as those in the following iPS cell production.

### (ii) iPS Cell

iPS cell can be produced by introducing a nuclear reprogramming substance into the somatic cell.

### (a) Somatic Cell Source

Examples of somatic cells that can be used as a starting material for the production of iPS cell may be any cell other than reproductive cell derived from a mammal (e.g., mouse or human). For example, keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), constrictive cells (e.g., smooth muscle cells), cells of the blood and the immune system (e.g., T lymphocytes), sense-related cells (e.g., rod cells), autonomic nervous system neurons (e.g., cholinergic neurons), sustentacular cells of sensory organs and peripheral neurons (e.g., satellite cells), nerve cells and glia cells of the central nervous system (e.g., astroglia cells), pigment cells (e.g., retinal pigment epithelial cells), progenitor cells thereof (tissue progenitor cells) and the like. There is no limitation on the degree of cell differentiation, and the like; even undifferentiated progenitor cells (including somatic stem cells) and finally differentiated mature cells can be used alike as sources of somatic cells in the present invention. Examples of undifferentiated progenitor cells include tissue stem cells (somatic stem cells) such as fat derived from stroma (stem) cells, neural stem cells, hematopoietic stem cells, mesenchymal stem cells, and dental pulp stem cells can be mentioned.

The choice of mammal individual as a source of collection of somatic cells is not particularly limited; however, when GSCLC as a final product is to be used for the treatment of disease such as human sterility and the like, it is preferable, from the viewpoint of prevention of graft rejection and/or GvHD, to collect the somatic cells from the patient's own cell or other person with the same or substantially the same HLA type as that of the patient. “Substantially the same HLA type” as used herein means that the HLA type of donor matches with that of patient to the extent that the transplanted cells, which have been obtained by inducing differentiation of iPS cells derived from the donor's somatic cells, can be engrafted when they are transplanted to the patient with use of immunosuppressant and the like. For example, it includes an HLA type wherein major HLAs (e.g., the three major loci of HLA-A, HLA-B and HLA-DR, the four loci further including HLA-Cw) are identical (hereinafter the same) and the like. When the PGC-like cells obtained are not to be administered (transplanted) to a human, but used as, for example, a source of cells for screening for evaluating the presence or absence of patient's drug susceptibility or adverse reactions, it is similarly necessary to collect the somatic cells from the patient or another person with the same genetic polymorphism correlating with the drug susceptibility or adverse reactions.

Somatic cells isolated from a mammal can be pre-cultured using a medium known per se suitable for their cultivation according to the choice of cells. Examples of such media include, but are not limited to, minimal essential medium (MEM) containing about 5 to 20% fetal bovine serum (FCS), Dulbecco's modified Eagle medium (DMEM), RPMI1640 medium, 199 medium, F12 medium, and the like. When a transfer reagent such as cationic liposome, for example, is used in bringing a cell into contact with nuclear reprogramming substances and another iPS cell establishment efficiency improver, it is sometimes preferable that the medium have been replaced in advance with a serum-free medium so as to prevent the transfer efficiency from decreasing.

### (b) Nuclear Reprogramming Substance

In the present invention, “a nuclear reprogramming substance” may be configured with any substance, such as a proteinous factor or a nucleic acid that encodes the same (including a form integrated in a vector), or a low molecular compound, as long as it is a substance (substances) capable of inducing an iPS cell from a somatic cell. When the nuclear reprogramming substance is a proteinous factor or a nucleic acid that encodes the same, preferable nuclear reprogramming substance is exemplified by the following combinations (hereinafter, only the names for proteinous factors are shown).

(1) Oct3/4, Klf4, c-Myc
(2) Oct3/4, Klf4, c-Myc, Sox2 (here, Sox2 is replaceable with Sox1, Sox3, Sox15, Sox17 or Sox18; Klf4 is replaceable with Klf1, Klf2 or Klf5; c-Myc is replaceable with T58A (active mutant), N-Myc or L-Myc)
(3) Oct3/4, Klf4, c-Myc, Sox2, Fbx15, Nanog, Eras, ECAT15-2, TclI, β-catenin (active mutant S33Y)
(4) Oct3/4, Klf4, c-Myc, Sox2, TERT, SV40 Large T antigen (hereinafter SV40LT)
(5) Oct3/4, Klf4, c-Myc, Sox2, TERT, HPV16 E6
(6) Oct3/4, Klf4, c-Myc, Sox2, TERT, HPV16 E7
(7) Oct3/4, Klf4, c-Myc, Sox2, TERT, HPV6 E6, HPV16 E7
(8) Oct3/4, Klf4, c-Myc, Sox2, TERT, Bmil
[For further information of the above-mentioned factors, see WO 2007/069666 (however, in the combination (2) above, for replacement of Sox2 with Sox18, and replacement of Klf4 with Klf1 or Klf5, see Nature Biotechnology, 26, 101-106 (2008)); for details of the combination “Oct3/4, Klf4, c-Myc, Sox2”, see also Cell, 126, 663-676 (2006), Cell, 131, 861-872 (2007) and the like. For details of the combination of “Oct3/4, Klf4, c-Myc, Sox2”, see also Cell, 126, 663-676 (2006), Cell, 131, 861-872 (2007) and the like. For details of the combination of “Oct3/4, Klf2 (or Klf5), c-Myc, Sox2”, see also Nat. Cell Biol., 11, 197-203 (2009). For details of the combination of “Oct3/4, Klf4, c-Myc, Sox2, hTERT, SV40LT”, see also Nature, 451, 141-146 (2008).)
(9) Oct3/4, Klf4, Sox2 (see also Nature Biotechnology, 26, 101-106 (2008))

### (10) Oct3/4, Sox2, Nanog, Lin28 (see Science, 318, 1917-1920 (2007))

(11) Oct3/4, Sox2, Nanog, Lin28, hTERT, SV40LT (see Stem Cells, 26, 1998-2005 (2008))
(12) Oct3/4, Klf4, c-Myc, Sox2, Nanog, Lin28 (see Cell Research (2008) 600-603)
(13) Oct3/4, Klf4, c-Myc, Sox2, SV40LT (see also Stem Cells, 26, 1998-2005 (2008))

### (14) Oct3/4, Klf4 (see Nature 454:646-650 (2008), Cell Stem Cell, 2:525-528 (2008))

(15) Oct3/4, c-Myc (see Nature 454:646-650 (2008))

### (16) Oct3/4, Sox2 (see Nature, 451, 141-146 (2008), WO 2008/118820)

### (17) Oct3/4, Sox2, Nanog (see WO 2008/118820)

### (18) Oct3/4, Sox2, Lin28 (see WO 2008/118820)

(19) Oct3/4, Sox2, c-Myc, Esrrb (Essrrb is replaceable with Esrrg. See Nat. Cell Biol., 11, 197-203 (2009))
(20) Oct3/4, Sox2, Esrrb (see Nat. Cell Biol., 11, 197-203 (2009))

### (21) Oct3/4, Klf4, L-Myc

### (22) Oct3/4, Nanog

### (23) Oct3/4

(24) Oct3/4, Klf4, c-Myc, Sox2, Nanog, Lin28, SV40LT (see Science, 324:797-801 (2009))

In the above-mentioned (1)-(24), a member of other Oct family, for example, Oct1A, Oct6 and the like can also be used instead of Oct3/4. In addition, a member of other Sox family, for example, Sox7 and the like can also be used instead of Sox2 (or Sox1, Sox3, Sox15, Sox17, Sox18). Furthermore, a member of other Lin family, for example, Lin28b and the like can also be used instead of Lin28.

Any combination that does not fall in (1) to (24) above but comprises all the constituents of any one of (1) to (22) and further comprises an optionally chosen other substance can also be included in the scope of “nuclear reprogramming substance” in the present invention. Provided that the somatic cell to undergo nuclear reprogramming is endogenously expressing one or more of the constituents of any one of (1) to (24) above at a level sufficient to cause nuclear reprogramming, a combination of only the remaining constituents excluding the one or more constituents can also be included in the scope of “nuclear reprogramming substance” in the present invention.

Of these combinations, at least one, preferably two or more, more preferably three or more selected from Oct3/4, Sox2, Klf4, c-Myc, Nanog, Lin28 and SV40LT are preferable nuclear reprogramming substances.

Among these combinations, when the obtained iPS cell is to be used for therapeutic purposes, a combination of 3 factors of Oct3/4, Sox2 and Klf4 (i.e., the above-mentioned (9)) is preferable. On the other hand, when the iPS cell is not to be used for therapeutic purposes (e.g., used as an investigational tool for drug discovery screening and the like), 4 factors of Oct3/4, Sox2, Klf4 and c-Myc, 5 factors of Oct3/4, Klf4, c-Myc, Sox2 and Lin28, or 6 factors further including Nanog (i.e., the above-mentioned (12)) or 7 factors further including SV40 Large T (i.e., the above-mentioned (24)), is preferable.

Furthermore, the above-mentioned combination with L-Myc instead of c-Myc is also a preferable example of a nuclear reprogramming substance.

Information on the mouse and human cDNA sequences of the aforementioned nuclear reprogramming substances is available with reference to the NCBI accession numbers mentioned in WO 2007/069666 (in the publication, Nanog is described as ECAT4. Mouse and human cDNA sequence information on Lin28, Lin28b, Esrrb, Esrrg and L-Myc can be acquired by referring to the following NCBI accession numbers, respectively); those skilled in the art are easily able to isolate these cDNAs.

| Name of gene | Mouse | Human |
|---|---|---|
| Lin28 | NM_145833 | NM_024674 |
| Lin28b | NM_001031772 | NM_001004317 |
| Esrrb | NM_011934 | NM_004452 |
| Esrrg | NM_011935 | NM_001438 |
| L-Myc | NM_008506 | NM_001033081 |

When a proteinous factor is used as a nuclear reprogramming substance, it can be prepared by inserting the cDNA obtained into an appropriate expression vector, transferring it into a host cell, culturing the cell, and recovering the recombinant proteinous factor from the cultured cells or a conditioned medium therefor. Meanwhile, when a nucleic acid that encodes a proteinous factor is used as a nuclear reprogramming substance, the cDNA obtained is inserted into a viral vector, plasmid vector, episomal vector or the like to construct an expression vector, which is subjected to the nuclear reprogramming step.

(c) Method for Introducing Nuclear Reprogramming Substance into Somatic Cell

Introduction of the nuclear reprogramming substance with a somatic cell, when the substance is a proteinaceous factor, can be achieved using a method known per se for protein transfer into a cell. In consideration of clinical application to human, iPS cell to be the starting material therefore is also preferably produced without gene manipulation.

Such methods include, for example, the method using a protein transfer reagent, the method using a protein transfer domain (PTD)- or cell penetrating peptide (CPP)-fusion protein, the microinjection method and the like. Protein transfer reagents are commercially available, including those based on a cationic lipid, such as BioPOTER Protein Delivery Reagent (Gene Therapy Systems), Pro-Ject™ Protein Transfection Reagent (PIERCE) and ProVectin (IMGENEX); those based on a lipid, such as Profect-1 (Targeting Systems); those based on a membrane-permeable peptide, such as Penetrain Peptide (Q biogene) and Chariot Kit (Active Motif), GenomONE (ISHIHARA SANGYO) utilizing HVJ envelope (inactive hemagglutinating virus of Japan) and the like. The transfer can be achieved per the protocols attached to these reagents, a common procedure being as described below. The nuclear reprogramming substance is diluted in an appropriate solvent (e.g., a buffer solution such as PBS or HEPES), a transfer reagent is added, the mixture is incubated at room temperature for about 5 to for 15 minutes to form a complex, this complex is added to cells after exchanging the medium with a serum-free medium, and the cells are incubated at 37° C. for one to several hours. Thereafter, the medium is removed and replaced with a serum-containing medium.

Developed PTDs include those using transcellular domains of proteins such as drosophila-derived AntP, HIV-derived TAT (Frankel, A. et al, Cell 55, 1189-93 (1988) or Green, M. & Loewenstein, P. M. Cell 55, 1179-88 (1988)), Penetratin (Derossi, D. et al, J. Biol. Chem. 269, 10444-50 (1994)), Buforin II (Park, C. B. et al. Proc. Natl Acad. Sci. USA 97, 8245-50 (2000)), Transportan (Pooga, M. et al. FASEB J. 12, 67-77 (1998)), MAP (model amphipathic peptide) (Oehlke, J. et al. Biochim. Biophys. Acta. 1414, 127-39 (1998)), K-FGF (Lin, Y. Z. et al. J. Biol. Chem. 270, 14255-14258 (1995)), Ku70 (Sawada, M. et al. Nature Cell Biol. 5, 352-7 (2003)), Prion (Lundberg, P. et al. Biochem. Biophys. Res. Commun. 299, 85-90 (2002)), pVEC (Elmquist, A. et al. Exp. Cell Res. 269, 237-44 (2001)), Pep-1 (Morris, M. C. et al. Nature Biotechnol. 19, 1173-6 (2001)), Pep-7 (Gao, C. et al. Bioorg. Med. Chem. 10, 4057-65 (2002)), SynBl (Rousselle, C. et al. Mol. Pharmacol. 57, 679-86 (2000)), HN-I (Hong, F. D. & Clayman, G L. Cancer Res. 60, 6551-6 (2000)), and HSV-derived VP22. CPPs derived from the PTDs include polyarginines such as 11R (Cell Stem Cell, 4, 381-384 (2009)) and 9R (Cell Stem Cell, 4, 472-476 (2009)).

A fused protein expression vector incorporating cDNA of a nuclear reprogramming substances and PTD or CPP sequence is prepared, and recombination expression is performed using the vector. The fused protein is recovered and used for transfer. Transfer can be performed in the same manner as above except that a protein transfer reagent is not added.

Microinjection, a method of placing a protein solution in a glass needle having a tip diameter of about 1 μm, and injecting the solution into a cell, ensures the transfer of the protein into the cell.

When the establishment efficiency of iPS cells is important, the nuclear reprogramming substance is also preferably used in the form of a nucleic acid encoding a proteinaceous factor rather than the proteinaceous factor itself. The nucleic acid may be a DNA or an RNA, or a DNA/RNA chimera. The nucleic acid may be double-stranded or single-stranded. Preferably, the nucleic acid is a double-stranded DNA, particularly cDNA.

cDNA of a nuclear reprogramming substance is inserted into an appropriate expression vector comprising a promoter capable of functioning in a host somatic cell. Useful expression vectors include, for example, viral vectors such as retrovirus, lentivirus, adenovirus, adeno-associated virus, herpes virus and Sendai virus, plasmids for the expression in animal cells (e.g., pA1-11, pXT1, pRc/CMV, pRc/RSV, pcDNAI/Neo) and the like.

The type of a vector to be used can be chosen as appropriate according to the intended use of the iPS cell to be obtained. Useful vectors include adenoviral vector, plasmid vector, adeno-associated viral vector, retroviral vector, lentiviral vector, Sendai viral vector, episomal vector and the like.

Examples of promoters used in expression vectors include the EF1α promoter, the CAG promoter, the SRα promoter, the SV40 promoter, the LTR promoter, the CMV (cytomegalovirus) promoter, the RSV (Rous sarcoma virus) promoter, the MoMuLV (Moloney mouse leukemia virus) LTR, the HSV-TK (herpes simplex virus thymidine kinase) promoter and the like, with preference given to the EF1a promoter, the CAG promoter, the MoMuLV LTR, the CMV promoter, the SRα promoter and the like.

The expression vector may contain as desired, in addition to a promoter, an enhancer, a polyadenylation signal, a selectable marker gene, a SV40 replication origin and the like. Examples of selectable marker genes include the dihydrofolate reductase gene, the neomycin resistant gene, the puromycin resistant gene and the like.

Nucleic acid as a nuclear reprogramming substance (reprogramming gene) may be incorporated on individual expression vectors, 2 or more kinds, preferably 2-3 kinds, of genes may be incorporated into one expression vector. The former case is preferable when using a retroviral or lentiviral vector that offers high gene transfer efficiency, and the latter is preferable when using a plasmid, adenoviral, or episomal vector and the like. Furthermore, an expression vector incorporating 2 or more kinds of genes, and other expression vector incorporating one gene alone can also be used in combination.

In the context above, when multiple reprogramming genes are integrated in one expression vector, these genes can preferably be integrated into the expression vector via a sequence enabling polycistronic expression. By using a sequence enabling polycistronic expression, it is possible to more efficiently express a plurality of genes integrated in one expression vector. Useful sequences enabling polycistronic expression include, the 2A sequence of foot-and-mouth disease virus (PLoS ONE 3, e2532, 2008, Stem Cells 25, 1707, 2007), the IRES sequence (U.S. Pat. No. 4,937,190) and the like, with preference given to the 2A sequence.

An expression vector harboring a nucleic acid which is a nuclear reprogramming substance can be introduced into a cell by a technique known per se according to the choice of the vector. In the case of a viral vector, for example, a plasmid containing the nucleic acid is introduced into an appropriate packaging cell (e.g., Plat-E cells) or a complementary cell line (e.g., 293-cells), the viral vector produced in the culture supernatant is recovered, and the vector is infected to a cell by a method suitable for the viral vector. For example, specific means using a retroviral vector are disclosed in WO2007/69666, Cell, 126, 663-676 (2006) and Cell, 131, 861-872 (2007). Specific means using a lentiviral vector is disclosed in Science, 318, 1917-1920 (2007). When PGC-like cell induced from iPS cell is utilized as a regenerative medicine for infertility treatment, gene therapy of germ cell and the like, since expression (reactivation) of reprogramming gene may increase the carcinogenic risk of germ cell or reproductive tissue regenerated from PGC-like cell derived from iPS cell, nucleic acid encoding a nuclear reprogramming substance is preferably expressed transiently, without being integrated into the chromosome of the cells. From this viewpoint, it is preferable to use an adenoviral vector, which is unlikely to be integrated into the chromosome, is preferred. Specific means using an adenoviral vector is disclosed in Science, 322, 945-949 (2008). Adeno-associated virus vector is unlikely to be integrated into the chromosome, and is less cytotoxic and less phlogogenic than adenoviral vectors, so that it is another preferred vector. Sendai virus vectors are capable of being stably present outside of the chromosome, and can be degraded and removed using an siRNA as required, so that they are preferably utilized as well. Useful Sendai virus vectors are described in J. Biol. Chem., 282, 27383-27391 (2007) or JP-B-3602058.

When a retroviral vector or a lentiviral vector is used, even if silencing of the transgene has occurred, it possibly becomes reactive; therefore, for example, a method can be used preferably wherein a nucleic acid encoding nuclear reprogramming substance is cut out using the Cre-loxP system, when becoming unnecessary. That is, with loxP sequences arranged on both ends of the nucleic acid in advance, iPS cells are induced, thereafter the Cre recombinase is allowed to act on the cells using a plasmid vector or adenoviral vector, and the region sandwiched by the loxP sequences can be cut out. Because the enhancer-promoter sequence of the LTR U3 region possibly upregulates a host gene in the vicinity thereof by insertion mutation, it is more preferable to avoid the expression regulation of the endogenous gene by the LTR outside of the loxP sequence remaining in the genome without being cut out, using a 3′-self-inactive (SIN) LTR prepared by deleting the sequence, or substituting the sequence with a polyadenylation sequence such as of SV40. Specific means using the Cre-loxP system and SIN LTR is disclosed in Chang et al., Stem Cells, 27: 1042-1049 (2009).

Meanwhile, being a non-viral vector, a plasmid vector can be transferred into a cell using the lipofection method, liposome method, electroporation method, calcium phosphate co-precipitation method, DEAF dextran method, microinjection method, gene gun method and the like. Specific means using a plasmid as a vector are described in, for example, Science, 322, 949-953 (2008) and the like.

When a plasmid vector, an adenovirus vector and the like are used, the transfection can be performed once or more optionally chosen times (e.g., once to 10 times, once to 5 times or the like). When two or more kinds of expression vectors are introduced into a somatic cell, it is preferable that these all kinds of expression vectors be concurrently introduced into a somatic cell; however, even in this case, the transfection can be performed once or more optionally chosen times (e.g., once to 10 times, once to 5 times or the like), preferably the transfection can be repeatedly performed twice or more (e.g., 3 times or 4 times).

Also when an adenovirus or a plasmid is used, the transgene can get integrated into chromosome; therefore, it is eventually necessary to confirm the absence of insertion of the gene into chromosome by Southern blotting or PCR. For this reason, like the aforementioned Cre-loxP system, it can be advantageous to use a means wherein the transgene is integrated into chromosome, thereafter the gene is removed. In another preferred mode of embodiment, a method can be used wherein the transgene is integrated into chromosome using a transposon, thereafter a transposase is allowed to act on the cell using a plasmid vector or adenoviral vector so as to completely eliminate the transgene from the chromosome. As examples of preferable transposons, piggyBac, a transposon derived from a lepidopterous insect, and the like can be mentioned. Specific means using the piggyBac transposon is disclosed in Kaji, K. et al., Nature, 458: 771-775 (2009), Woltjen et al., Nature, 458: 766-770 (2009).

Another preferable non-integration type vector is an episomal vector, which is capable of self-replication outside of the chromosome. Specific means using an episomal vector is disclosed by Yu et al., in Science, 324, 797-801 (2009). Where necessary, an expression vector may be constructed by inserting a reprogramming gene into an episomal vector having loxP sequences placed in the same orientation on the 5′ and 3′ sides of a vector component essential for the replication of the episomal vector, and transferred to a somatic cell.

Examples of the episomal vector include a vector comprising as a vector component a sequence derived from EBV, SV40 and the like necessary for self-replication. The vector component necessary for self-replication is specifically exemplified by a replication origin and a gene that encodes a protein that binds to the replication origin to control the replication; examples include the replication origin oriP and the EBNA-1 gene for EBV, and the replication origin ori and the SV40 large T antigen gene for SV40.

The episomal expression vector comprises a promoter that controls the transcription of a reprogramming gene. The promoter used may be as described above. The episomal expression vector may further contain as desired an enhancer, a polyadenylation signal, a selection marker gene and the like, as described above. Examples of the selection marker gene include the dihydrofolate reductase gene, the neomycin resistance gene and the like.

An episomal vector can be transferred into a cell using, for example, the lipofection method, liposome method, electroporation method, calcium phosphate co-precipitation method, DEAE dextran method, microinjection method, gene gun method and the like. Specifically, for example, methods described in Science, 324: 797-801 (2009) and elsewhere can be used.

Whether or not the vector component necessary for the replication of the reprogramming gene has been removed from the iPS cell can be confirmed by performing a Southern blot analysis or PCR analysis using a part of the vector as a probe or primer, with the episome fraction isolated from the iPS cell as a template, and determining the presence or absence of a band or the length of the band detected. The episome fraction can be prepared by a method obvious in the art; for example, methods described in Science, 324: 797-801 (2009) can be used.

When the nuclear reprogramming substance is a low-molecular-weight compound, the substance can be introduced into a somatic cell by dissolving the substance at a suitable concentration in an aqueous or non-aqueous solvent, adding the solution to a medium suitable for the culture of somatic cell isolated from human or mouse (e.g., minimum essential medium (MEM), Dulbecco's modified Eagle medium (DMEM), RPMI1640 medium, 199 medium, F12 medium and the like containing about 5-20% fetal bovine serum such that the concentration of a nuclear reprogramming substance is sufficient to cause nuclear reprogramming in the somatic cell and free of cytotoxicity, and culturing the cells for a given period. While the concentration of the nuclear reprogramming substance varies depending on the kind of the nuclear reprogramming substance to be used, it is appropriately selected from the range of about 0.1 nM-about 100 nM. The contact period is not particularly limited as long as it is sufficient for achieving nuclear reprogramming of the cell. Generally, they may be co-existed in the medium until positive colony emerges.

### (d) Establishment Efficiency Improving Substance for iPS Cell

Since the iPS cell establishment efficiency has been low, various substances that improve the efficiency have recently been proposed one after another. It can be expected, therefore, that the iPS cell establishment efficiency will be increased by bringing another establishment efficiency improver, in addition to the aforementioned nuclear reprogramming substance, into contact with the transfer subject somatic cell.

Examples of the iPS cell establishment efficiency improving substance include, but are not limited to, histone deacetylase (HDAC) inhibitors [e.g., low-molecular inhibitors such as valproic acid (VPA) (Nat. Biotechnol., 26(7):795-797 (2008), trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29 mer shRNA Constructs against HDAC1 (OriGene) and the like), and the like], DNA methyl transferase inhibitors (e.g., 5-azacytidine) (Nat. Biotechnol., 26(7):795-797 (2008)), G9a histone methyl transferase inhibitors [for example, low-molecular inhibitors such as BIX-01294 (Cell Stem Cell, 2:525-528 (2008)), and nucleic acid-based expression inhibitors such as siRNAs and shRNAs (Cell Stem Cell, 3, 475-479 (2008)) against G9a], L-channel calcium agonist (e.g., Bayk8644) (Cell Stem Cell, 3, 568-574 (2008)), p53 inhibitor (e.g., siRNA and shRNA to p53, UTF1 (Cell Stem Cell, 3, 475-479 (2008)), Wnt Signaling (e.g., soluble Wnt3a) (Cell Stem Cell, 3, 132-135 (2008)), 2i/LIF (2i is inhibitor of mitogen-activated protein kinase signalling and glycogen synthase kinase-3, PloS Biology, 6(10), 2237-2247 (2008)) and the like. The nucleic acid-based expression inhibitors mentioned above may be in the form of expression vectors harboring a DNA that encodes an siRNA or shRNA.

Of the aforementioned constituents of nuclear reprogramming substances, SV40 large T, for example, can also be included in the scope of iPS cell establishment efficiency improvers because it is an auxiliary factor unessential for the nuclear reprogramming of somatic cells. While the mechanism of nuclear reprogramming remains unclear, it does not matter whether auxiliary factors, other than the factors essential for nuclear reprogramming, are deemed nuclear reprogramming substances or iPS cell establishment efficiency improvers. Hence, because the somatic cell nuclear reprogramming process is taken as an overall event resulting from contact of a nuclear reprogramming substance and an iPS cell establishment efficiency improver with a somatic cell, it does not always seem to be essential for those skilled in the art to distinguish between the two.

An iPS cell establishment efficiency improver can be contacted with a somatic cell as mentioned above for each of (a) when the substance is a proteinous factor and (b) when the substance is a nucleic acid encoding the proteinous factor, or (c) when the substance is a low-molecular-weight compound.

An iPS cell establishment efficiency improver may be contacted with a somatic cell simultaneously with a nuclear reprogramming substance, and either one may be contacted in advance, as far as the iPS cell establishment efficiency from a somatic cell improves significantly compared with the efficiency obtained in the absence of the improver. In an embodiment, for example, when the nuclear reprogramming substance is a nucleic acid that encodes a proteinous factor and the iPS cell establishment efficiency improver is a chemical inhibitor, the iPS cell establishment efficiency improver can be added to the medium after the cell is cultured for a given length of time after the gene transfer treatment, because the nuclear reprogramming substance involves a given length of time lag from the gene transfer treatment to the mass-expression of the proteinous factor, whereas the iPS cell establishment efficiency improver is capable of rapidly acting on the cell. In another embodiment, for example, when the nuclear reprogramming substance and iPS cell establishment efficiency improver are both used in the form of a viral vector or plasmid vector, both may be simultaneously transferred into the cell.

### (e) Improving the Establishment Efficiency by Culture Conditions

The iPS cell establishment efficiency can further be improved by culturing the cells under hypoxic conditions in the nuclear reprogramming process for somatic cells. As mentioned herein, the term “hypoxic conditions” means that the ambient oxygen concentration as of the time of cell culture is significantly lower than that in the atmosphere. Specifically, conditions involving lower oxygen concentrations than the ambient oxygen concentrations in the 5-10% CO₂/95-90% air atmosphere, which is commonly used for ordinary cell culture, can be mentioned; examples include conditions involving an ambient oxygen concentration of 18% or less. Preferably, the ambient oxygen concentration is 15% or less (e.g., 14% or less, 13% or less, 12% or less, 11% or less and the like), 10% or less (e.g., 9% or less, 8% or less, 7% or less, 6% or less and the like), or 5% or less (e.g., 4% or less, 3% or less, 2% or less and the like). The ambient oxygen concentration is preferably 0.1% or more (e.g., 0.2% or more, 0.3% or more, 0.4% or more and the like), 0.5% or more (e.g., 0.6% or more, 0.7% or more, 0.8% or more, 0.95% or more and the like), or 1% or more (e.g., 1.1% or more, 1.2% or more, 1.3% or more, 1.4% or more and the like).

While any method of creating a hypoxic state in a cellular environment can be used, the easiest way is to culture cells in a CO₂ incubator permitting adjustments of oxygen concentration, and this represents a suitable case. CO₂ incubators permitting adjustment of oxygen concentration are commercially available from various manufacturers (e.g., CO₂ incubators for hypoxic culture manufactured by Thermo scientific, Ikemoto Scientific Technology, Juji Field, Wakenyaku etc.).

The time of starting cell culture under hypoxic conditions is not particularly limited, as far as iPS cell establishment efficiency is not prevented from being improved compared with the normal oxygen concentration (20%). The start time may be before or after the somatic cell is contacted with the nuclear reprogramming substance, or at the same time as the contact, or after the contact, it is preferable, for example, that the culture under hypoxic conditions be started just after the somatic cell is contacted with the nuclear reprogramming substance, or at a given time interval after the contact [e.g., 1 to 10 (e.g., 2, 3, 4, 5, 6, 7, 8 or 9) days].

The duration of cultivation of cells under hypoxic conditions is not particularly limited, as far as iPS cell establishment efficiency is not prevented from being improved compared with the normal oxygen concentration (20%); examples include, but are not limited to, periods of 3 days or more, 5 days or more, for 7 days or more or 10 days or more, and 50 days or less, 40 days or less, 35 days or less or 30 days or less and the like. Preferred duration of cultivation under hypoxic conditions varies depending on ambient oxygen concentration; those skilled in the art can adjust as appropriate the duration of cultivation according to the oxygen concentration used. In an embodiment of the present invention, if iPS cell candidate colonies are selected with drug resistance as an index, it is preferable that a normal oxygen concentration be restored from hypoxic conditions before starting drug selection.

Furthermore, preferred starting time and preferred duration of cultivation for cell culture under hypoxic conditions also vary depending on the choice of nuclear reprogramming substance used, iPS cell establishment efficiency at normal oxygen concentrations and the like.

After contacting a nuclear reprogramming substance (and iPS cell establishment efficiency improving substance), the cell can, for example, be cultured under conditions suitable for cultivation of ES cells. In the case of mouse cells, generally, the cultivation is carried out with the addition of leukemia inhibitory factor (LIF) as a differentiation suppression factor to an ordinary medium. On the other hand, in the case of human cells, it is desirable that basic fibroblast growth factor (bFGF) and/or stem cell factor (SCF) be added in place of LIF. Typically, the cell is cultured in the co-presence of mouse embryonic fibroblasts (MEFs) treated with radiation or an antibiotic to terminate the cell division, as feeder cells. Usually, STO cells and the like are commonly used as MEFs; for induction of an iPS cell, however, the SNL cell (McMahon, A. P. & Bradley, A. Cell 62, 1073-1085 (1990)) and the like are commonly used. Co-culture with these feeder cells may be started before contact with a nuclear reprogramming substance, at the time of the contact, or after the contact (e.g., 1-10 days later).

A candidate colony of iPS cells can be selected in two ways: methods with drug resistance and reporter activity as indicators, and methods based on macroscopic examination of morphology. As an example of the former, a colony positive for drug resistance and/or reporter activity is selected using a recombinant cell wherein the locus of a gene highly expressed specifically in pluripotent cells (e.g., Fbx15, Nanog, Oct3/4 and the like, preferably Nanog or Oct3/4) is targeted by a drug resistance gene and/or a reporter gene. Examples of such recombinant cells include MEFs derived from a mouse having the βgeo (which encodes a fusion protein of β-galactosidase and neomycin phosphotransferase) gene knocked in to the Fbx15 gene locus (Takahashi & Yamanaka, Cell, 126, 663-676 (2006)), and MEFs derived from a transgenic mouse having the green fluorescent protein (GFP) gene and the puromycin resistance gene integrated in the Nanog gene locus [Okita et al., Nature, 448, 313-317 (2007)]. On the other hand, methods for selecting a candidate colony by macroscopic examination of morphology include, for example, the method described by Takahashi et al. in Cell, 131, 861-872 (2007). Although the methods using reporter cells are convenient and efficient, colony selection by macroscopic examination is desirable from the viewpoint of safety when iPS cells are prepared for therapeutic purposes in humans. When 3 factors of Oct3/4, Klf4 and Sox2 are used as the nuclear reprogramming substance, the number of the established clones decreases, but almost all resulting colonies are iPS cells having high quality comparable to that of ES cell. Therefore, iPS cell can be established efficiently even without using a reporter cell.

The identity of the cells of the selected colony as iPS cells can be confirmed by positive responses to Nanog (or Oct3/4) reporters (puromycin resistance, GFP positivity and the like), as well as by the visible formation of an ES cell-like colony, as described above; however, to ensure greater accuracy, it is possible to perform tests such as analyzing the expression of various ES-cell-specific genes, and transplanting the selected cells to a mouse and confirming teratoma formation.

(iii) Naive Human ES and iPS Cell

Conventional human ES cell induced from blastocyst phase embryo has biological (morphological, molecular and functional) properties vastly different from those of mouse ES cell. Mouse pluripotent stem cell may be present in two functionally distinct states: LIF-dependent ES cell and bFGF-dependent epiblast stem cells (EpiSC). Molecular analysis suggests that the pluripotency state of human ES cell is not that of mouse ES cell, but rather similar to that of mouse EpiSC. Recently, human ES and iPS cell (also called naive human ES and iPS cell) in a mouse ES cell-like pluripotency state have been established by ectopically inducing Oct3/4, Sox2, Klf4, c-Myc and Nanog in the presence of LIF (see Cell Stem Cells, 6:535-546, 2010), or ectopically inducing Oct3/4, Klf4 and Klf2 by combining LIF and GSK3β and ERK1/2 pathway inhibitor (see Proc. Natl. Acad. Sci. USA, online publication doi/10.1073/pnas.1004584107). Since these naive human ES and iPS cells have immature pluripotency as compared to conventional human ES and iPS cells, they may be preferable as starting materials for the present invention.

(2) Differentiation Induction from Pluripotent Stem Cell into EpiLC

Examples of the basic medium for differentiation induction include, but are not limited to, Neurobasal medium, Neural Progenitor Basal medium, NS-A medium, BME medium, BGJb medium, CMRL 1066 medium, minimum essential medium (MEM), Eagle MEM medium, αMEM medium, Dulbecco's modified Eagle medium (DMEM), Glasgow MEM medium, Improved MEM Zinc Option medium, IMDM medium, Medium 199 medium, DMEM/F12 medium, ham medium, RPMI 1640 medium, Fischer's medium, and a mixed medium of these and the like.

The medium may be a serum-containing medium or serum-free medium. Preferably, a serum-free medium is used. The serum-free medium (SFM) means a medium free of an untreated or unpurified serum, and therefore, a medium containing purified blood-derived component or animal tissue-derived component (growth factor and the like) can be mentioned. The concentration of the serum (e.g., fetal bovine serum (FBS), human serum and the like) may be 0-20%, preferably 0-5%, more preferably 0-2%, most preferably 0% (that is, serum-free). SFM may or may not contain an optional serum replacement. Examples of the serum replacement include albumin (e.g., lipid-rich albumin, albumin substitute recombinant albumin and the like, plant starch, dextran and protein hydrolysate etc.), transferrin (or other iron transporter), fatty acid, insulin, collagen precursor, trace element, 2-mercaptoethanol, 3′-thioglycerol or a substance containing an equivalent of these and the like as appropriate. Such serum replacement can be prepared, for example, by the method described in WO 98/30679. To simplify more, a commercially available product can be utilized. Examples of such commercially available substance include Knockout (trade mark) Serum Replacement (KSR), Chemically-defined Lipid concentrated, and Glutamax (Invitorogen).

The medium may contain other additives known per se. The additive is not particularly limited as long as EpiLC equivalent to epiblast cell before intestinal invagination is produced by the method of the present invention. For example, growth factors (e.g., insulin and the like), polyamines (e.g., putrescine and the like), minerals (e.g., sodium selenite and the like), saccharides (e.g., glucose and the like), organic acids (e.g., pyruvic acid, lactic acid and the like), amino acids (e.g., non-essential amino acid (NEAA), L-glutamine and the like), reducing agents (e.g., 2-mercaptoethanol and the like), vitamins (e.g., ascorbic acid, d-biotin and the like), steroids (e.g., [beta]-estradiol, progesterone and the like), antibiotics (e.g., streptomycin, penicillin, gentamicin and the like), buffering agents (e.g., HEPES and the like), nutrition additives (e.g., B27 supplement, N2 supplement, StemPro-Nutrient Supplement and the like) can be mentioned. Each additive is preferably contained in a concentration range known per se.

In the production method of EpiLC in the present invention, pluripotent stem cell may be cultured in the presence or absence of feeder cells. Feeder cells are not particularly limited as long as EpiLC can be produced by the method of the present invention. A feeder cell known per se can be used for culturing pluripotent stem cells such as ESC, iPSC and the like. For example, fibroblasts (mouse embryonic fibroblast, mouse fibroblast strain STO and the like) can be mentioned. Feeder cell is preferably inactivated by a method known per se, for example, a treatment with radiation (gamma ray and the like), an anti-cancer agent (mitomycin C and the like) and the like. However, in a preferable embodiment of the present invention, pluripotent stem cells are cultured under feeder-free conditions.

The medium for differentiation induction of human pluripotent stem cells into EpiLC (medium A) contains a basal medium and activin A as an essential additive. The concentration of activin A in the medium for differentiation induction is, for example, not less than about 5 ng/ml, preferably not less than about 10 ng/ml, more preferably not less than about 15 ng/ml and, for example, not more than about 40 ng/ml, preferably not more than about 30 ng/ml, more preferably not more than 25 ng/ml.

The medium A preferably further contains bFGF and/or KSR. Basic FGF and KSR present in an effective concentration range remarkably increases induction efficiency of EpiLC. The concentration of bFGF is, for example, not less than about 5 ng/ml, preferably not less than about 7.5 ng/ml, more preferably not less than about 10 ng/ml and, for example, not more than about 30 ng/ml, preferably not more than about 20 ng/ml, more preferably not more than about 15 ng/ml. The concentration of KSR is, for example, not less than about 0.1 w/w %, preferably not less than about 0.3 w/w %, more preferably not less than about 0.5 w/w % and, for example, not more than about 5 w/w %, preferably not more than about 3 w/w %, more preferably not more than about 2 w/w %.

In a particularly preferable embodiment, medium A contains a basic medium, as well as activin A, bFGF and KSR. An appropriate concentration of these components can be selected from the range of about 10-about 30 ng/ml, preferably about 15-about 25 ng/ml, for activin A; about 7.5-about 20 ng/ml, preferably about 10-about 15 ng/ml for bFGF; and about 0.3-about 3 w/w %, preferably about 0.5-about 2 w/w %, for KSR.

The source of activin A and bFGF to be contained in medium A is not limited, and may be isolated and purified from any mammalian (e.g., human, mouse, monkey, swine, rat, dog and the like) cells. It is preferable to use activin A and bFGF which are allogeneic to the pluripotent stem cell subjected to culture. Activin A and bFGF may be chemically synthesized, may be biochemically synthesized using a cell-free translation system, or may be produced from a transformant that has a nucleic acid encoding each protein. Recombinant products of activin A and bFGF are commercially available.

A culture vessel used for inducing pluripotent stem cells into EpiLC is not particularly limited, and flask, tissue culture flask, dish, petri dish, tissue culture dish, multidish, microplate, microwell plate, multiplate, multiwall plate, microslide, chamber slide, petri dish, tube, tray, culture bag, and roller bottle can be mentioned. The culture vessel may be cell adhesive. A cell adhesive culture vessel may be coated with any cell adhesion substrate such as extracellular matrix (ECM) and the like for the purpose of improving adhesiveness of the culture vessel surface to the cells. The cell adhesion substrate may be any substance aiming at adhesion of pluripotent stem cells or feeder cells (when used). As the cell adhesion substrate, collagen, gelatin, poly-L-lysine, poly-D-lysine, poly-L-ornithine, laminin, and fibronectin and a mixture thereof, such as Matrigel and lysed cellular membrane preparations can be mentioned (Klimanskaya I et al 2005. Lancet 365: p 1636-1641).

For culturing, human pluripotent stem cells are seeded on the above-mentioned culture vessel to a cell density of, for example, about 10⁴-10⁵ cells/cm², preferably about 2-8×10⁴ cells/cm², and cultured under an atmosphere of 1-10% CO₂/99-90% air in an incubator at about 30-40° C., preferably about 37° C., for less than 3 days, preferably 2 days (e.g., 48±12 hr, preferably 48±6 hr). As a result of the culture, cells having a flat epiblast-like structure appear uniformly.

The fact of differentiation into EpiLC can be confirmed, for example, by analyzing the expression level of the marker gene of EpiLC and/or pluripotent stem cell by RT-PCR. The EpiLC in the present invention means a cell in an epiblast-like (pre-gastrulation epiblast-like) state of E5.5 to E6.0. More particularly, EpiLC is defined as a cell having either or both of the following properties:

(1) an increase in at least one gene expression selected from Fgf5, Wnt3 and Dnmt3b, compared to pluripotent stem cell before m differentiation induction,
(2) a decrease in at least one gene expression selected from Gata4, Gata6, Sox17 and Blimp1, compared to pluripotent stem cell before differentiation induction.

Therefore, the fact of differentiation into EpiLC can be confirmed by measuring the expression level of at least one selected from Fgf5, Wnt3 and Dnmt3b and/or at least one selected from Gata4, Gata6, Sox17 and Blimp1, in the cells obtained by culturing, and comparing the expression levels of the pluripotent stem cell before differentiation induction.

More preferably, the EpiLC of the present invention has the following properties:

(1) sustained gene expression of Oct3/4;
(2) decreased gene expression of Sox2 and Nanog compared to that of pluripotent stem cell before differentiation induction;
(3) increased gene expression of Fgf5, Wnt3 and Dnmt3b compared to pluripotent stem cell before differentiation induction; and
(4) decreased gene expression of Gata4, Gata6, Sox17 and Blimp1 compared to that of pluripotent stem cell before differentiation induction.

As mentioned above, in a preferable embodiment, the medium A of the present invention contains activin A, bFGF and KSR. Therefore, the present invention also provides a reagent kit for differentiation induction of pluripotent stem cells into EpiLC, which contains activin A, bFGF and KSR. These components may be provided in the form of being dissolved in water or a suitable buffer, provided as a freeze-dry powder and can also be used upon dissolution in a suitable solvent when in use. Also, these components may be placed in a kit each as a single reagent, or two or more kinds thereof may be mixed and provided as a single reagent as long as they do not adversely influence each other.

The present inventors have succeeded for the first time in producing EpiLC having properties equivalent to those of pre-gastrulation epiblast cells though transiently. Epiblast is also a precursor of somatic cell lineages other than the germline cells. Thus, the EpiLC thus obtained can be used as starting cell material for inducing not only the germline cells but also various other cell lineages. It may also be useful for investigating the genetic and epigenetic mechanism underlying epiblast differentiation in ICM, which is an important subject but scarcely understood in pluripotent cell biology. As an intermediate for a particular lineage, the derivation of EpiLC from ESC or iPSC is a very direct process, and provides a new strategy for reconstituting the lineage specification in vitro.

(3) Differentiation Induction of EpiLC into PGC-Like Cell

Differentiation of the thus-obtained EpiLC into PGC-like cells can be induced by culturing in the presence of BMP4 and LIF (Cell, 137, 571-584(2009)). Therefore, a second aspect of the present invention relates to a method for producing PGC-like cells from pluripotent stem cells via EpiLC obtained by the method of the above-mentioned (2). Therefore, the method includes

I) a step of producing EpiLC from pluripotent stem cells according to any method described in the above-mentioned (2); and
II) a step of culturing the EpiLC obtained in step I) in the presence of BMP4 and LIF.

The basal medium for differentiation induction in step II), the basal medium exemplified to be used in step I) is preferably used in the same manner. The medium may contain the same additives as those exemplified for use in step I) as long as a PGC-like cell capable of contributing to normal spermatogenesis can be produced by the method of the present invention.

The medium may be a serum-containing medium or serum-free medium (SFM). Preferably, a serum-free medium is used. The concentration of the serum (e.g., fetal bovine serum (FBS), human serum and the like) may be 0-20%, preferably 0-5%, more preferably 0-2%, most preferably 0% (i.e., serum-free). SFM may or may not contain any serum replacement such as KSR and the like.

A medium for differentiation induction of EpiLC into a PGC-like cell (medium B) contains, as an essential additive of a basic medium, bone morphogenetic protein 4 (BMP4) and a leukemia inhibitory factor (LIF). The concentration of BMP4 is, for example, not less than about 100 ng/ml, preferably not less than about 200 ng/ml, more preferably not less than about 300 ng/ml. The concentration of BMP4 is, for example, not more than about 1,000 ng/ml, preferably not more than about 800 ng/ml, more preferably not more than 600 ng/ml. The concentration of LIF is, for example, not less than about 300 U/ml, preferably not less than about 500 U/ml, more preferably not less than about 800 U/ml. The concentration of LIF is, for example, not more than about 2,000 U/ml, preferably not more than about 1,500 U/ml, more preferably not more than 1,200 U/ml.

Medium B further preferably contains at least one additive selected from stem cell factor (SCF), bone morphogenic protein8b (BMP8b) and epidermal growth factor (EGF). When SCF, BMP8b and EGF are present within an effective concentration range, the period of maintaining PGC-like cell in a Blimp1- and Stella-positive state is markedly extended. The concentration of SCF is, for example, not less than about 30 ng/ml, preferably not less than about 50 ng/ml, more preferably not less than about 80 ng/ml. The concentration of SCF is, for example, not more than about 200 ng/ml, preferably not more than about 150 ng/ml, more preferably not more than about 120 ng/ml. The concentration of BMP8b is, for example, not less than about 100 ng/ml, preferably not less than about 200 ng/ml, more preferably not less than about 300 ng/ml. The concentration of BMP8b is, for example, not more than about 1,000 ng/ml, preferably not more than about 800 ng/ml, more preferably not more than 600 ng/ml. The concentration of EGF is, for example, not less than about 10 ng/ml, preferably not less than about 20 ng/ml, more preferably not less than about 30 ng/ml. The concentration of EGF is, for example, not more than about 100 ng/ml, preferably not more than about 80 ng/ml, more preferably not more than about 60 ng/ml.

In a particularly preferable embodiment, medium B contains a basic medium, as well as BMP, LIF, SCF, BMP8b and EGF. The concentration of these components can be appropriately selected from the range of about 200-800 ng/ml, preferably about 300-600 ng/ml, for BMP4; about 500-1500 U/ml, preferably about 800-1,200 U/ml, for LIF; about 50-150 ng/ml, preferably about 80-120 ng/ml, for SCF; about 200-800 ng/ml, preferably about 300-600 ng/ml, for BMP8b; and about 20-80 ng/ml, preferably about 30-60 ng/ml, for EGF.

The source of BMP4, LIF, SCF, BMP8b and EGF to be contained in medium B is not particularly limited, and may be isolated and purified from any mammalian (e.g., human, mouse, monkey, swine, rat, dog and the like) cells. It is preferable to use BMP4, LIF, SCF, BMP8b and EGF which are allogeneic to the EpiLC subjected to culture. BMP4, LIF, SCF, BMP8b and EGF may be chemically synthesized, may be biochemically synthesized using a cell-free translation system, or may be produced from a transformant that has a nucleic acid encoding each protein. Recombinant products of BMP4, LIF, SCF, BMP8b and EGF are commercially available.

For culturing, EpiLC is seeded in a cell non-adhesive or low adhesive culture vessel known per se to a cell density of, for example, about 3-10×10⁴ cells/ml, preferably about 4-8×10⁴ cells/ml, and cultured under an atmosphere of 1-10% CO₂/99-90% air in an incubator at about 30-40° C., preferably about 37° C., for about 4-10 days, preferably about 4-8 days, more preferably about 4-6 days, further preferably about 4 days.

The fact of differentiation into PGC-like cells can be confirmed, for example, by analyzing the expression of BLIMP1 by RT-PCR and the like. Where necessary, expression of other gene and cell surface antigen can also be examined. As other gene, Stella can be mentioned. When pluripotent stem cells having a fluorescence protein gene under control of Blimp1- and/or Stella-promoter is used as a starting material, the fact of differentiation into PGC-like cells can be confirmed by FACS analysis. When pluripotent stem cells derived from human or other non-mouse mammal such as ESC or iPSC and the like do not have an appropriate transgenic reporter, the fact of differentiation into PGC-like cells is preferable confirmed by FACS analysis and the like using one or more kinds of cell surface antigens specifically expressed in PGC-like cells. As the cell surface antigen, for example, SSEA-1 and integrin-β3 are preferable.

The cell population containing pluripotent stem cell-derived PGC-like cells, which is produced by the aforementioned steps I) and II), is may be a purified population of PGC-like cells, and one or more kinds of cells may co-exist besides PGC-like cell. As used herein, the “PGC-like cell” is defined as a cell that shows an increase in the expression of Blimp1 and/or Stella compared to EpiLC before differentiation induction, can contribute to the normal spermatogenesis, and does not form teratoma when transplanted to an immunodeficient mouse. As mentioned above, when PGC-like cell is induced using a pluripotent stem cells having a fluorescence protein gene under control of BLIMP1- and/or Stella-promoter as a starting material, Blimp1- and/or Stella-positive PGC-like cell can be easily isolated and purified by sorting by a cell sorter the cell population obtained in the aforementioned step II). PGC-like cell can also be isolated and purified by FACS using, as a marker, a reporter under control of a gene (e.g., Nanog) whose expression increases with Blimp1 and Stella.

### 2. Maintenance/Expansion of PGC-Like Cell

The PGC-like cell obtained as mentioned above can be expanded prior to differentiation into a spermatogenic stem cell-like cell. As a medicament that supports amplification of PGC, forskoline and retinoic acid (RA) signaling agonist are known. Forskoline activates adenyate cyclase and increases the intracellular cAMP level. The present inventors previously found that phosphodiesterase 4 (PDE4) inhibitors support expansion of PGC-like cells. PDE4 inhibitors increase intracellular cAMP levels by a mechanism (inhibiting hydrolysis of cAMP) different from that of forskoline. Thus, the present inventors succeeded in synergistically (up to about 50 times) promoting expansion of PGC-like cells by a combined use of PDE4 inhibitor and forskoline (EMBO J., 36(13): 1888-1907 (2017)).

Therefore, maintenance/expansion of PGC-like cell can be performed by culturing, for example, PGC-like cells on d4-d10, preferably d4-d8, more preferably d4-d6, further preferably about d4, wherein the day of the start of differentiation induction from EpiLC is d0, in the presence of a PDE4 inhibitor, preferably, in the further presence of forskoline. As the basic medium, the medium exemplified with regard to differentiation induction from PSC into EpiLC can be used similarly. It is preferable to add a serum or serum replacement to the medium. As the kind and addition concentration of the serum or serum replacement used here, those exemplified with regard to differentiation induction from PSC into EpiLC can be used similarly. In addition, the medium may contain other additives known per se. Such additive is not particularly limited as long as it can support maintenance/expansion of PGC-like cell, and those exemplified with regard to differentiation induction from PSC into EpiLC can be used similarly. Examples of the medium to be used for this step include, but are not limited to, GMEM medium containing 10% Knockout Serum Replacement (KSR), 2.5% fetal bovine serum (FCS), 0.1 mM NEAA, 1 mM sodium pyruvate, 0.1 mM 2-mercaptoethanol, 100 U/ml penicillin, 0.1 mg/ml streptomycin, and 2 mM L-glutamine, and the like.

The PDE4 inhibitor to be added to the above-mentioned medium is not particularly limited as long as it is a substance that can inhibit enzyme activity of PDE4, namely, hydrolysis activity of cAMP. It is preferably a selective inhibitor of PDE4 (which does not inhibit not only enzyme other than phosphodiesterase (PDE) but also PDEs other than PDE4). Examples thereof include, but are not limited to, ibudilast, S-(+)-rolipram, rolipram, GSK256066, cilomilast and the like.

The concentration of the PDE4 inhibitor is, for example, not less than about 0.1 μM, preferably not less than about 0.5 μM, more preferably not less than about 1 μM. The concentration of the PDE4 inhibitor is, for example, not more than about 100 μM, preferably not more than about 50 μM, more preferably not more than 30 μM. In a preferable embodiment, the concentration of the PDE4 inhibitor can be appropriately selected from the range of about 0.5-50 μM, preferably about 1-30 μM.

The concentration of forskoline to be added to the above-mentioned medium is, for example, not less than about 0.1 μM, preferably not less than about 0.5 μM, more preferably not less than about 1 μM, and the concentration of forskoline is, for example, not more than about 100 μM, preferably not more than about 50 μM, more preferably not more than 30 μM. In a preferable embodiment, the concentration of forskoline can be appropriately selected from the range of about 0.5-50 μM, preferably about 1-30 μM.

The medium for maintenance/expansion of PGC-like cell preferably further contains SCF. The concentration of SCF is, for example, not less than about 30 ng/ml, preferably not less than about 50 ng/ml, more preferably not less than about 80 ng/ml. The concentration of SCF is, for example, not more than about 200 ng/ml, preferably not more than about 150 ng/ml, more preferably not more than about 120 ng/ml. In a preferable embodiment, the concentration of SCF can be appropriately selected from the range of about 50-150 ng/ml, preferably about 80-120 ng/ml.

In a particularly preferable embodiment, the medium for maintenance/expansion of PGC-like cell contains 10 μM PDE4 inhibitor, 10 μM forskoline and 100 ng/ml SCF. When used in combination with other PGC proliferation stimulation factor, dedifferentiation of the PGC-like cell into EGC may be promoted. Thus, it is sometimes preferable not to add LIF to the medium for maintenance/expansion of PGC-like cell.

In a method for maintenance/expansion of PGC-like cell, the PGC-like cell may be cultured in the presence or absence of feeder cells. The kind of feeder cell is not particularly limited and a feeder cell known per se can be used. For example, fibroblast (mouse embryo [symbol] fibroblast, mouse fibroblast strain STO and the like) can be mentioned. The feeder cell is preferably inactivated by a method known per se, for example, a treatment with radiation (gamma ray and the like), anti-cancer agent (mitomycin C and the like) and the like. When the feeder cell is fragile to PDE4 inhibitor and/or forskoline, it is desirable to passage culture several generations of the feeder cell in advance in the presence of these additives to be acclimated to the additives.

The culture vessel to be used for maintenance/expansion of the PGC-like cell is not particularly limited and, for example, those exemplified with regard to differentiation induction from PSC into EpiLC can be used similarly.

In this culture, the PGC-like cells are plated on a culture vessel (with feeder cells seeded in advance) to a cell density of, for example, about 10⁴-10⁵ cells/cm², preferably about 2-8×10⁴ cells/cm², and cultured under an atmosphere of 1-10% CO₂/99-90% air in an incubator at about 30-40° C., preferably about 37° C., for 3-9 days, preferably 4-8 days, more preferably 5-7 days. As a result of the culture, flat colonies are formed, Blimp1 and Stella are strongly and continuously expressed, characteristics of motile cell accompanying filopodium and lamellipodium are shown, and the property of PGC in motile phase is maintained.

3. Production of Spermatogenic Stem Cell-Like Cell from Primordial Germ Cell-Like Cell (PGCLC)

### (1) Formation of Reconstituted Testis (Step (1))

In this step, for example, PGCLC obtained by the above-mentioned method is co-cultured with gonad somatic cells in suspension culture to obtain reconstituted testis. When PGCLC to be used is a non-uniform cell population, for example, it is preferable to use an SSEA-1 positive and integrin-β3 positive cell fraction isolated by FACS. As PGCLC, PGC-like cells on d4-d10, preferably d4-d8, more preferably d4-d6, further preferably about d4, wherein the day of the start of differentiation induction from EpiLC is d0, can be used. As the PGCLC, PGCLC maintained/expanded for 3-9 days, preferably 4-8 days, more preferably 5-7 days, by the method described in detail in the above-mentioned 2. may also be used.

The “gonad” here refers to a structure composed of a germ cell and a somatic cell in support thereof. In the mother fetus, it is formed by the time when sexual differentiation between male and female begins in fetal (pup) primordial germ cells (PGC) (12.5 days after fertilization (E12.5) in mouse). PGC differentiates into gamete (spermatozoon or oocyte) while being surrounded by somatic cells of the gonad which is characteristic of each of male and female. In the present invention, to mimic the cellular environment when PGC becomes pro-spermatogonium, the gonad at this stage (e.g., E12.0-E13.0, preferably about E12.5 for mouse) is used. The gonad can be collected by a method known per se from a mammal allogeneic to PGCLC to be co-cultured. Examples of the method for isolating a somatic cell from the gonad include, but are not limited to, a method including dissociating the gonad into single cells by a trypsin treatment and the like, resuspending the cells in a medium or an isotonic buffer such as phosphate buffered saline (PBS) and the like, and removing cell surface marker positive cells of PGC, for example, SSEA-1 positive cells, using FACS or MACS, and the like.

Then, PGCLC and gonad somatic cells are co-cultured in suspension culture. Here, “suspension culture” means culturing the cell or cell aggregate of interest without adhesion to the bottom surface of the culture vessel. Culturing in a state where, even if a cell or cell aggregate is in contact with the bottom surface, the cell or cell aggregate floats in the culture medium when the culture medium is slightly shaken is also included in the suspension culture. For suspension culture, it is preferable to use, as a culture vessel, for example, a plastic dish with an untreated bottom surface or one coated with a coating agent (poly(2-hydroxyethylmethacrylate) etc.) for preventing adhesion of cells to a substrate. For example, 10³-10⁵, preferably about 10⁴, PGCLC, and 2×10³-1×10⁶, preferably 2-10×10⁴, further preferably about 4×10⁴, gonad somatic cells per one reconstituted testis are added to a medium at a ratio of, for example, PGCLC:gonad somatic cell=1:2-1:15, 1:2-1:10, preferably 1:3-1:5, more preferably about 1:4, and static culture can be performed. In the medium to be used in this step, the medium exemplified with regard to differentiation induction from pluripotent stem cell into EpiLC can be similarly used as the basic medium. It is preferable to add a serum or serum replacement to the medium. As the kind and addition concentration of the serum or serum replacement used here, those exemplified with regard to differentiation induction from pluripotent stem cell into EpiLC can be used similarly. In addition, the medium may contain other additives known per se. Such additive is not particularly limited as long as PGCLC and gonad somatic cell can self organize to form an aggregate (reconstituted testis) mimicking the testis and those exemplified with regard to differentiation induction from pluripotent stem cell into EpiLC can be used similarly. Examples of the medium to be used for this reconstituted testis formation step (present step (1)) include, but are not limited to, αMEM medium containing 10% Knockout Serum Replacement (KSR) and the like. When maintenance/expansion culture of PGCLC is performed prior to this step, a medium having the same composition as the medium used for the maintenance/expansion culture can also be used.

The suspension culture of this step (1) is performed, for example, under an atmosphere of 1-10% CO₂/99-90% air in an incubator at about 30-40° C., preferably about 37° C., for about 1-5 days, preferably about 1-3 days, more preferably about 2-3 days, further preferably about 2 days.

### (2) Induction of Spermatogenic Stem Cell-Like Cell (Step (2))

In this step, the reconstituted testis obtained in step (1) is subjected to gas/liquid interfacial culture to induce spermatogenic stem cell-like cell in the reconstituted testis. The “gas/liquid interfacial culture” refers to a culture method including seeding cells on a porous membrane of a cell culture insert, and, with the upper side as a gas phase, supplying a medium from below through the membrane. The reconstituted testis obtained in step (1) is placed on a porous membrane of a culture insert inserted into a culture vessel (e.g., 24 well plate), the medium is added to the lower side of the membrane and gas/liquid interfacial culture is performed. The medium to be used may be the same as that in step (1). The culture is performed under an atmosphere of 1-10% CO₂/99-90% air in an incubator at about 30-40° C., preferably about 34-37° C., for about 1-8 weeks, preferably about 2-8 weeks, more preferably about 2-4 weeks, further preferably about 3 weeks. When maintenance/expansion of PGCLC is performed prior to the induction of spermatogenic stem cell-like cell, the culture period of this step can also be set to, for example, about 1-2 weeks, preferably about 10-14 days. In preferable one embodiment, the culture temperature is about 34° C. throughout this step. In another embodiment, a method including culturing at about 37° C. for the first two weeks and thereafter at 34° C. can also be used.

During this step, in the reconstituted testis, seminiferous tubule-like structures begin to appear from about day 4, and they assemble to form a network by about day 7. PGCLC-derived cells outside the seminiferous tubule-like structure disappear, and the majority of PGCLC-derived cells are localized inside the seminiferous tubule-like network. Thereafter, the reconstituted testis is maintained stably during which it further develops the seminiferous tubule-like network, and the number of PGCLC-derived cells inside the m network increases more. At day 14, most PGCLC-derived cells are present in the luminal compartment of the seminiferous tubules, and at day 21, many of them are found in the basal compartment.

On day 14, many of PGCLC-derived cells are positive for DDX4(MVH) which is a germ cell marker that begins to be expressed in gonad PGC; however, they are negative for PLZF(ZBTB16) which is a spermatogenic stem cell marker that begins to be expressed in perinatal period in pro-spermatogonium. At day 21, most of the PGCLC-derived cells are DDX4 positive, and some of them are DDX4/PLZF double positive. When culture is continued for a long term, some of the PGCLC-derived cells become positive for SCP3 which is an important marker of meiosis initiation; however, meiosis is not completed under the conditions of this step.

4. Production of Germline Stem Cell-Like Cell (GSCLC) from Reconstituted Testis Containing Spermatogenic Stem Cell-Like Cell

The present invention also provides a method for producing a cell having properties equivalent to those of a germline stem cell (GSC) which is a long-term culture cell line of a spermatogenic stem cell, namely, a germline stem cell-like cell (GSCLC), from a reconstituted testis containing a spermatogenic stem cell-like cell obtained as mentioned above. The method includes dissociating a spermatogenic stem cell-like cell into a single cell from a reconstituted testis containing a spermatogenic stem cell-like cell obtained by the above-mentioned steps (1) and (2) by, for example, an enzyme treatment (e.g., DISPASE, hyaluronidase, trypsin) and a pipetting operation, and culturing the cell under conditions capable of inducing GSC from a spermatogenic stem cell. A spermatogenic stem cell-like cell can be separated from a gonad somatic cell-derived cell by FACS and the like using, for example, a cell surface marker such as CD9, SSEA1, INTEGRINβ1, INTEGRINα6, KIT, GFRα1 and the like, or when PGCLC is a reporter cell, using the reporter as an index. The cell can be conveniently enriched by performing several passages using the difference in the adhesiveness to a culture vessel between somatic cell and spermatogenic stem cell-like cell. For example, using a culture vessel coated with gelatin, collagen, Matrigel, laminin and the like, dissociated cells derived from reconstituted testis are seeded and non-adherent cells are passaged in a new culture vessel every, for example, 6 to 24 hours, preferably about 12 hours. Since somatic cells with high adhesiveness remain on the surface of the culture vessel and are gradually removed, spermatogenic stem cell-like cells are enriched.

The medium to be used in this step is not particularly limited as long as it can support induction GSC from spermatogenic stem cells. For example, a medium containing glial cell-derived neurotrophic factor (GDNF) and leukemia inhibitory factor (LIF), and preferably further containing epithelial cell growth factor (EGF) and/or basic fibroblast growth factor (bFGF) can be mentioned. For more detailed composition of the medium, the descriptions of WO 2004/092357 and Kanatsu-Shinohara, M. et al., Biol. Reprod. 69, 612-616 (2003) can be referred to. More specifically, as preferable one embodiment, the GSC/GSCLC medium described in the below-mentioned Examples can be mentioned.

The spermatogenic stem cell-like cell enriched as mentioned above is preferably cultured in the presence of a feeder cell during culture in this step. As the feeder cell, for example, mouse fetal fibroblast (MEF) and the like are preferably used. When the enriched spermatogenic stem cell-like cells are seeded as PGCLC-derived cells at a cell density of not less than about 10³ cells in a culture vessel in which m feeder cells are seeded in advance, GSC-like colonies are expanded in the first two weeks. When the diameter of colony reaches not less than about 500 μm, the colony can be subcultured in a new culture vessel.

The enriched spermatogenic stem cell-like cells are cultured, for example, under 1-10% CO₂/99-90% air atmosphere in an incubator at about 30-40° C., preferably about 37° C., for about 2 weeks or more, preferably about 2 months or more.

The GSC-like cells (GSCLC) obtained as mentioned above have the following properties:

(a) derived from isolated PSC,
(b) having expression levels equivalent to those of GSC as regards
(i) a gene selected from the group consisting of Ddx4, Daz1, Gfra1, Ret, Piwil2, Itga6, Kit, Plzf, Piwil4 and Id4,
(ii) a surface marker selected from the group consisting of CD9, SSEA1, INTEGRINβ1, INTEGRINα6, KIT and GFRα1, and
(iii) a transcription factor selected from the group consisting of PLZF and ID4,
(c) can be maintained or expanded at a proliferation rate equivalent to that of GSC,
(d) when GSCLC is transplanted into an adult testis,
(i) a proportion of seminiferous tubule having GFRα1-positive cells to seminiferous tubule with colonized transplanted cells is equivalent to that when GSC is transplanted, and
(ii) a proportion of seminiferous tubule having SCP3-positive cells to seminiferous tubule with colonized transplanted cells is lower than that when GSC is transplanted,
(e) microinsemination with a sperm cell obtained by transplantation of the GSCLC into an adult testis produces a normal offspring.

Thus, the GSCLC of the present invention is equivalent to conventionally-known GSCs in key gene, expression levels of cell surface marker and transcription factor, cell proliferation rate, emergence rate of spermatogonial/spermatogenic stem cell marker (GFRα1)-positive seminiferous tubule when transplanted in adult testis, and ability to produce normal offspring, whereas different from GSC induced from spermatogenic stem cell collected from a body in the derivation from isolated PSC, and low emergence rate of meiocyte (SCP3-positive) when transplanted in adult testis, as compared to GSC.

### 5. Use of GSCLC of the Present Invention

The thus established GSCLC derived from pluripotent stem cell can be used for various purposes. For example, since GSCLC transplanted into the testis of a recipient animal can certainly contribute to spermatogenesis in the testis, particularly adult testis, and creation of a healthy offspring, it can be used for the treatment of sterility, or hereditary diseases of reproductive tissues.

GSCLC can be transplanted into the testis according to the methods described in WO 2004/092357 and Biol. Reprod., 69:612-616 (2003) by using GSCLC instead of GSC. As a result of the transplantation, a sperm cell (spermatozoon or round spermatid) differentiated from GSCLC can fertilize an oocyte by the ICSI or ROSI method known per se, and the obtained embryo (e.g., 2-cell phase embryo) is transplanted into the uterus or oviduct of a pseudopregnant host, whereby an offspring can be obtained.

The GSCLC (including cell population containing GSCLC; hereinafter the same) of the present invention is mixed with a pharmaceutically acceptable carrier and the like according to a conventional means and produced as a parenteral preparation, preferably, injection, suspension or drip transfusion. Examples of the pharmaceutically acceptable carrier that can be contained in the parenteral preparation include an aqueous solution for injection such as saline, isotonic solution (e.g., D-sorbitol, D-mannitol, sodium chloride and the like) containing glucose and other auxiliary agents, and the like. The agent of the present invention may be blended with, for example, a buffering agent (e.g., phosphate buffer, sodium acetate buffer), a soothing agent (e.g., benzalkonium chloride, procaine hydrochloride and the like), a stabilizer (e.g., human serum albumin, polyethylene glycol and the like), a preservative, an antioxidant and the like.

When the agent of the present invention is prepared as an aqueous suspension, GSCLC is suspended in one of the above-mentioned aqueous solutions at a cell density of about 1.0×10⁶-about 1.0×10⁷ cells/mL.

The agent of the present invention can be preserved at a low temperature under the conditions typically used for low temperature preservation of stem cells, and can be thawed immediately before use.

The thus-obtained preparation is stable and of lower toxicity, and thus can be safely administered to mammals such as human and the like. While the administration method is not particularly limited, it is preferable to administer the preparation as an injection or drip into a seminiferous tubule. For male sterile patients, for example, about 1.0×10⁵-about 1×10⁷ cells of the agent in the amount of GSCLC is generally conveniently administered 1 or 2 to 10 times at about 1-2 week intervals.

The present invention demonstrates for the first time the in vitro reconstitution of the male germ cell differentiation determination pathway from an inner cell mass to a spermatogenic stem cell. Such in vitro system reflecting the development processes not only promotes elucidation of the detailed development mechanism of germ cell but also will promote elucidation of the mechanism of the onset of sterility and hereditary diseases.

The present invention is hereinafter described in further detail by means of the following examples, to which, however, the scope of the present invention is not limited.

### EXAMPLE

### Example 1

### (Outline of Method)

### Culture of ESC and Induction of PGCLC

Embryonic stem cells (ESC) (C57BL/6×129/SvJcl) (Ohta et al., 2000) having AAG transgene were cultured using N2B27 medium containing 2i (PD0325901: 0.4 μM [Stemgent]; CHIR99021: 3 μM (Stemgent)) and LIF (1,000 U/ml) on a well (Hayashi et al., 2011; Hayashi and Saitou, 2013; Ying et al., 2008) coated with poly-L-ornithine and laminin (20 ng/mL). EpiLCs were induced from 1.0×10⁵ ESCs on a well of a 12 well plate coated with human plasma fibronectin (16.7 g/mL) using an epiblast-like cell (EpiLC) medium (N2B27 containing activin A [20 ng/mL], bFGF [12 ng/mL] and knockout serum replacement [KSR] [1%] [Thermo Fisher Scientific]). The EpiLC medium was exchanged every day. PGCLCs were induced from 2.0×10³ EpiLCs in a low cell binding U-bottom 96 well lipidure-coat plate under floating conditions using PGCLC medium (GMEM [Invitrogen] containing 15% KSR, 0.1 mM NEAA, 1 mM sodium pyruvate, 0.1 mM 2-mercaptoethanol, 100 U/mL penicillin, 0.1 mg/mL streptomycin, and 2 mM L-glutamine [100 ng/mL], BMP4 [500 ng/mL] [R&D Systems], LIF [1,000 U/mL] [Invitrogen], SCF [100 ng/mL] [R&D Systems], and EGF [50 ng/mL] [R&D Systems] were used (4-6 days)).

### Production and Culture of Reconstituted Testes

Using α-minimum essential medium containing 10% KSR (α-MEM) (Invitrogen), d4 PGCLCs (10,000 cells/reconstituted testis) collected by FACS and E12.5 gonad (ICR) somatic cells (40,000 cells/reconstituted testis) collected by MACS (see magnetic-activated cell sorting) were allowed to aggregate (37° C., 5% CO₂) in a low cell binding U-bottom 96 well of lipidure-coat plate under floating conditions. After suspension culture for 2 days, the aggregates were transferred using glass capillary to a well of a Falcon permeable support for a 24 well plate having a 0.4 μm transparent PET membrane (Corning). For gas/liquid interfacial culture (Sato et al., 2011), each well was supplemented with 350 μL α-MEM-10% KSR (Sato et al., 2011). The medium was exchanged every week.

Induction of GSCLC from Reconstituted Testes and Induction of GSC from Neonatal Testes

Reconstituted testes were immersed in dissociation buffer (DMEM containing DISPASE [1 mg/mL] [Invitrogen] and hyaluronidase [1 mg/mL] [Sigma, H3506]) for 10 min, then incubated in 0.05% trypsin-0.53 mM EDTA for 15 min with periodical (every 5 min) pipetting, quenched in DMEM containing 10% FBS, and successively dissociated into single cells by accurate pipetting. The cell suspension was centrifuged at 1,200 rpm for 5 min and the supernatant was removed. The cell pellets were suspended in GSC/GSCLC culture medium containing a growth factor (see below), and the cells were seeded on a culture plate coated with 0.1% (w/v) gelatin (cells/reconstituted testes were transferred to 24 well plate). Since somatic cells are more easily bound to a culture plate, they were removed as much as possible by repeatedly passaging the somatic cells every 12 hr. After 2 or 3 passages, the remaining AAG(+) cells were transferred on a plate containing MEF in a GSC/GSCLC medium containing a growth factor (see below). When about 1×10³ or more AAG(+) cells/well were seeded, GSCLC colony expanded in the first 2 weeks. When a GSCLC colony with a diameter exceeding about 500 μm was developed, it was passaged in a new well and GSCLC cell line was established in about 2 months. A control GSC cell line was induced from neonatal (129/Sv×C57BL/6 having AAG) testes at P7 by using essentially the same procedure. The medium for GSC/GSCLC culture was the medium described in Kanatsu-Shinohara et al. (2003) with partial modification: StemPro-34 SFM (Invitrogen) was supplemented with Stem Pro supplement, 1% FBS, 1×Gluta-MAX-1 (Invitrogen), 1×minimum essential medium (MEM) vitamin solution (Sigma), 5 mg/mL AlbuMAX-II (Invitrogen), 5×10⁻⁵ M 2-mercaptoethanol, 1×MEM non-essential amino acid solution (Invitrogen), 30 μg/mL pyruvic acid, 1×ITS-G (Invitrogen), 100 U/mL penicillin, 0.1 mg/mL streptomycin, and growth factor (recombinant rat GDNF [10 ng/mL] [R&D Systems], human bFGF [10 ng/mL] [Invitrogen], LIF/ESGRO [10³ U/mL] [Invitrogen], and mouse EGF [20 ng] [Invitrogen]).

### Accession Number

The accession numbers for RNA-seq data of GSC1, 2 and GSCLC1-4, SC3-seq data of GSC1, 2, GSCLC1 and 4, WGBS data of GSC1 and GSCLC 1-3, and WGBS data of GSC2 and GSCLC4 reported in the present specification are respectively NCBI GEO: GSE76245, GSE87341, DDBJ: DRA004241 and DRA005141.

### Animal

All animal experiments were performed based on the ethical guidelines of Kyoto University. Acro/Act-EGFP (AAG) transgenic mouse (gift from M. Okabe) (Nakanishi et al., 1999; Okabe et al., 1997) largely maintained the background of C57BL/6. W/W^{v} (WB×C57BL/6), BDF1 (C57BL/6×DBA/2), ICR and 129/SvJcl mice were purchased from SLC (Hamamatsu, Japan). The midday of the day when the vaginal plug was confirmed was taken as embryonic day (E)0.5.

### Fluorescence-Activated Cell Sorting (FACS)

Aggregates containing d4 PGCLCs were washed with PBS, dissociated into single cells by treating with 0.05% trypsin-0.53 mM EDTA for 7 min, quenching with DMEM containing 10% FBS and successively pipetting. The cell suspension was passed through a nylon cell strainer (diameter 40 μm) to remove cell aggregates. Flow-through cells were centrifuged at 1200 rpm for 5 min, and the supernatant was removed. The cell pellets were suspended in PBS containing 0.1% bovine serum albumin (BSA) (Thermo Fisher Scientific, San Diego, Calif.) and incubated on ice for 15 min with an anti-integrin β3 antibody (BioLegend) and an anti-SSEA1 antibody (eBioscience) respectively conjugated with PE and Alexa Fluor 647. After washing with PBS-0.1% BSA, the cells were suspended in the same buffer (1×10⁶ cells/ml) and sorted by a flow cytometer (AriaIII:BD Biosciences).

For expression analysis of surface markers of GSCLC/GSC, GSCLCs/GSCs were dissociated into single cells by treating with 0.05% trypsin-0.53 mM EDTA for 4 min, quenching with DMEM containing 10% FBS and thereafter pipetting. The cell suspension was centrifuged at 1,200 rpm for 5 min and the supernatant was removed. The cell pellets were suspended in PBS containing 0.1% BSA (about 1×10⁶ cells were suspended in 200 μl of PBS-0.1% BSA). A half amount of the suspension was stained with a primary antibody (Antibodies) on ice for 15 min. The remaining half was used to establish an unstained control. After washing with PBS-0.1% BSA, respective suspensions were stained with a secondary antibody (Antibodies) on ice for 15 min. The cells were suspended in PBS-0.1% BSA and analyzed by a flow cytometer (AriaIII; BD Biosciences). In all analyses, AAG(+) cells were selected before use.

### Magnetic-Activated Cell Sorting (MACS)

Male gonads of E12.5 were isolated in DMEM containing 10% FBS, washed with PBS, and dissociated into single cells with 0.05% trypsin-0.53 mM EDTA. The cell suspension was passed through a nylon cell strainer (diameter 70 μm) (Falcon) to remove cell aggregates. Flow through cells were centrifuged at 1200 rpm for 5 min, and the supernatant was removed. The cell pellets were suspended in PBS containing 0.5% BSA and 2 mM EDTA, and incubated on ice for 15 min with an anti-SSEA1 antibody conjugated with magnetic beads (Miltenyi Biotec, Bergisch, Gladbach, Germany) (Antibodies). The cell suspension was washed with PBS-0.5% BSA-0.2 mM EDTA and then applied to an MS column (Miltenyi Biotec). Flow through cells after removal of most SSEA-1 positive PGC were washed with PBS-0.5% BSA-0.2 mM EDTA, suspended in αMEM-10% KSR, and allowed to aggregate with PGCLC to produce reconstituted testes.

Transplantation of PGCLC and GSCLC into Testes

GSCs and GSCLCs were dissociated into single cells by incubating with 0.05% trypsin-0.53 mM EDTA for 4 min, and quenched with DMEM containing 10% FBS. The cell suspension was passed through a nylon cell strainer (diameter 40 μm) (Falcon) to remove cell aggregates. Flow through cells were centrifuged at 1200 rpm for 5 min, and the supernatant was removed. The pellets were suspended in GSC/GSCLC medium without a growth factor at a concentration of 2.5×10⁷ cells/ml. For transplantation of PGCLCs, 10,000 cells of d4 PGCLC collected by FACS were suspended in 5 μl of GSC/GSCLC medium. As described before (Brinster and Avarbock, 1994; Brinster and Zimmermann, 1994), about 5 μl of each donor cell suspension was injected into rete testes of an adult WBB6F1 W/W^{v} (8-week-old) recipient with a pipette provided with a syringe. The transplanted testes were analyzed at 8 to 10 weeks post-transplantation.

### Intra-Cytoplasmic Sperm Injection (ICSI) and Round Spermatid Injection (ROSI) in Cytoplasm

Horse chorionic gonadotropin (5 IU) was injected, and 5 IU of human chorionic gonadotropin (hCG) was injected twice 48 hr thereafter to induce superovulation in BDF1 female. 12 hr after hCG injection, cumulus-oocyte complexes were collected from the oviduct, and oocytes were treated with 0.1% hyaluronidase to be liberated from cumulus cells. In ICSI, spermatozoa were injected into the cytoplasm of an MII oocyte using a Piezo-actuated micromanipulator (Kimura and Yanagimachi, 1995). For ROSI, oocytes that received a round spermatid were cultured for 1 hr in KSOM medium containing 5 mM SrCl2 and 2 mM EGTA (Kishigami and Wakayama, 2007). The 2-cell embryo after ICSI or ROSI was transferred into the oviduct of a 0.5dpc pseudopregnant ICR female by standard procedure.

### Histology and Immunofluorescence (IF) Analysis

For histological analysis, testes with transplanted cells were fixed with Bouin fixative for 48 hr, washed three times with 70% ethanol, dehydrated with serial concentrations of ethanol, and embedded in paraffin. The tissue was cut to a thickness of 7 μm. After deparaffinizing with xylene (3 times) and stepwise ethanol series, the sections were stained with hematoxylin and eosin.

For IF analysis, reconstituted testes were fixed on ice with 4% para-formaldehyde (PFA) on ice for 2 hr, washed three times with PBS, replaced with serial concentrations of sucrose solution (15%, 30%), embedded in OCT compound (Sakura, Tokyo, Japan), frozen, and then cut to a thickness of 10 μm at −20° C. After air drying, the sections were washed three times with PBS, incubated in blocking buffer (PBS containing 5% BSA and 0.1% Triton X-100) for 30 min, and then reacted in a staining buffer (PBS containing 1% BSA and 0.1% Triton X-100) containing primary antibody (Antibodies) at 4° C. for 2 hr. After washing three times with PBS, the samples were incubated for 1 hr in a staining buffer containing secondary antibody (Antibodies) and 1 μg/ml DAPI. The sections were washed three times with PBS and mounted on Vectashield mounting medium (Vector Laboratories). All samples were analyzed with a confocal microscope (Olympus FV1000).

For IF analysis of GSC/GSCLC, GSCs/GSCLCs cultured on cover slip were fixed with 3% PFA for 10 min at room temperature, then washed three times with PBS, and successively permealized in methanol at −30° C. for 2 min. The samples were washed twice with PBS, blocked with PBS containing 10% BSA for 30 min at room temperature, and incubated with a solution (PBS containing 0.1% BSA) containing a combination of primary antibodies (antibody) (PBS containing 0.1% BSA) at room temperature for 2 hr. The samples were washed three times with PBS and incubated for 1 hr with a solution containing a combination of the primary antibodies (Antibodies) and 1 μg/ml DAPI. The sections were washed three times with PBS and mounted on Vectashield mounting medium (Vector Laboratories). All samples were analyzed with a confocal microscope (Zeiss LSM780). The antibodies used in this experiment are shown in Table 1.

TABLE 1

| Name | Description | Company | Product # | Concentration | IF/FACS |
|---|---|---|---|---|---|
| Primary antibody | | | | | |
| GFP | Chicken | Abcam ® | CM-AB13970 | 1:500 | IF |
| | polyclonal | | | | |
| GFP | Rat monoclonal | Nacalai Tesque | 04404-84 | 1:250 | IF |
| SOX9 | Rabbit polyclonal | MILLIPORE | ab5535 | 1:10000 | IF |
| DDX4 | Mouse | Abcam ® | ab27591 | 1:200 | IF |
| | monoclonal | | | | |
| SCP3 | Mouse | Abcam ® | ab97672 | 1:500 | IF |
| | monoclonal | | | | |
| GFRa1 | Goat polyclonal | R&D System | BAF560 | 1 μg/ml | IF |
| GATA4 | Goat polyclonal | Santa Cruz | sc4237 | 1:100 | IF |
| PLZF | Rabbit polyclonal | Santa Cruz | sc-22839 | 1:40 | IF |
| ID4 | Human | Abnova | H00003400-B01P | 1:200 | IF |
| | polyclonal | | | | |
| DAPI | — | WAKO | 049-18801 | 1 μg/ml | IF |
| Conjugated antibody | | | | | |
| CD49f(Itga6)-APC | Mouse/Human | Biolegend | 313615 | 1:50 | FACS |
| CD15(SSEA-1)-Alexa | Mouse/Human | Biolegend | 125607 | 1:20 | FACS |
| Fluor ® 647 | | | | | |
| CD9-APC | Mouse | eBioscience ™ | 17-0091 | 1:50 | FACS |
| | monoclonal | | | | |
| CD29(Itgβ1)-Alexa | Hamster | BD | 562153 | 1:50 | FACS |
| Fluor ® 647 | monoclonal | Pharmingen ™ | | | |
| CD117(KIT:2B8)-PE | Mouse | Affymetrix | 17-1171-81 | 1:50 | FACS |
| | monoclonal | eBioscience | | | |
| Primary antibody | | | | | |
| GFRa-1 | Mouse | R&D System | MAB560 | 25 ng/ml | FACS |
| | monoclonal | | | | |
| Isotype control | | | | | |
| PE Rat IgG2b κ | Rat polyclonal | Biolegend | 400635 | 1.50 | FACS |
| ARC Rat IgG2a κ | Rat polyclonal | Biolegend | 400511 | 1:50 | FACS |
| Alexa Fluor647 | Hamster | BD | 562110 | 1:50 | FACS |
| Hamster IgM, λ1 | polyclonal | Pharmingen ™ | | | |
| Secondary antibody | Fluorescence | | | | |
| Goat Anti-Chicken IgY | FITC | Abcam ® | Ab46969 | 1:250 | IF |
| Goat Anti Rabbit IgG | Alexa Fluor ® 568 | Invitrogen | A11011 | 1:250 | IF |
| Goat Anti-Mouse IgG | Alexa Fluor ® 633 | Invitrogen | A21052 | 1:250 | IF/FACS |
| Donkey Anti-Chicken | Alexa Fluor ® 488 | Abcam | ab63507 | 1:250 | IF |
| IgY | | | | | |
| Donkey Anti-Rat IgG | Alexa Fluor ® 488 | ThermoFisher | A-21208 | 1:250 | IF |
| | | Scientific | | | |
| Donkey Anti-Goat IgG | Alexa Fluor ® 568 | ThermoFisher | A-11057 | 1:250 | IF |
| | | Scientific | | | |
| Donkey Anti-Rabbit | Alexa Fluor ® 647 | ThermoFisher | A-31573 | 1:250 | IF |
| IgG | | Scientific | | | |

### Alkaline Phosphatase (AP) Staining

d4 PGCLCs directly cultured under GSC culture conditions were washed twice with PBS and fixed with 4% para-formaldehyde on ice for 1 hr. After washing with PBS containing 0.1% Tween20 (PBST), colonies derived from d4 PGCLC were incubated in AP buffer at room temperature. The AP buffer was prepared by sequentially dissolving 5 mg of naphthol AS-MX disodium phosphate (Sigma) and 10 mg of Fast Red TR salt (Sigma) in 0.5 ml of N,N-dimethylformamide (Sigma), followed by filtration. The reaction was terminated at an appropriate time point with PBST.

### Karyotype Analysis

GSCs/GSCLCs were cultured in a medium containing 60 ng/ml demecorsin (Sigma) for 6 hr, dissociated into single cells by incubating in 0.05% trypsin-0.53 mM EDTA for 4 min, and quenched with DMEM containing 10% FBS. The cells were then swollen with a hypotonic solution (75 mM KCL) and fixed by repeated treatments (3 times) with Carnoy fixative. The fixed cells were spread on a glass slide (Matsunami) washed with ethanol, and stained with DAPI. Karyotype images were obtained by analysis with a confocal microscope (Olympus FV1000) and the number of chromosomes was counted.

qPCR

RNA of GSCs/GSCLCs collected by FACS for AAG fluorescence was extracted and purified using the RNeasy Micro Kit (Qiagen, Hilden, Germany) according to the manufacturer's instructions. Purified total RNA was reverse transcribed with SuperscriptIII (Invitrogen) to generate first-strand cDNA, which was used for qPCR analysis with Power SYBER Green (ABI, Foster City, Calif.). The primer sequences used are shown in Table 2.

TABLE 2

| | |
|---|---|
| Forward | Reverse |

qPCR

| | | |
|---|---|---|
| Arbp | CAAAGCTGAAGCAAAGG | AATTAAGCAGGCTGAC |
| | AAGAG | TTGGTTG |
| Ppia | TTACCCATCAAACCATT | AACCCAAAGAACTTCA |
| | CCTTCTG | GTGAGAGC |
| Oct4 | GATGCTGTGAGCCAAGG | GGCTCCTGATCAACAG |
| | CAAG | CATCAC |
| T | ATCAGAGTCCTTTGCTA | GTTACAATCTTCTGGC |
| | GGTAG | TATGC |
| Dmrt2 | TAGACAGATGCGCAAAA | GCCCTTTACTGAGAGA |
| | GACCT | CGTGAG |
| Sohlh1 | CCAGAGTTTAGTTTGCT | ATACAAACTCCACTCA |
| | GGGGA | CAGCCC |
| H19 | TAAACCTCTTTGGCAAT | ATGGAACTGCTTCCAG |
| | GCTGC | ACTAGG |
| Eomes | ACTCACTGCCTTCAATT | CCCTTTCTTTCTGATC |
| | CCTTG | CTCCTG |
| Ovol2 | GTGCCTTGAAATGCTTC | GAGAAGTTTCACACGA |
| | CCAT | ACATTTCATTTA |
| Tel1 | TGGCCTCACTAGAACAA | CTCGGTCAAGGATGGA |
| | GAGG | AGC |
| Tex19.1 | CGTGTCAGTGTTCAGTG | GTCAACTAGTGCCTCA |
| | TTTGG | GAGTCC |
| Hoxb2 | CCATCGACTTGCAGTTT | ATGTATCCACGAGTGG |
| | CCCTA | AGAAGG |
| Hoxb3 | GGTTGTTGTGTTTCCTG | AGGAGCTGAGTTCATG |
| | TCATGT | CCTTTT |
| Hoxb5 | TATGGGGATAGTCTGGG | CAGATTTCACACGTAG |
| | TCAGG | CACAGC |
| Hoxb6 | ACTTGATGTCTCCTGGA | CGAATCTACCATTGAA |
| | AGCAG | CCGTGC |
| Hoxb7 | TGTAAGCCCTCTTTGAA | TGCTACTGGGAAGTAT |
| | GCTGT | GGGGTA |
| Hoxb8 | TGGTCTGTTTCCTTTGA | TTACGGCGTGAATAGG |
| | ACGTG | CAGTTT |
| Hoxb9 | AGAAGACTCAGAGTGGG | CGATGTTTGCCTCTTT |
| | GACTT | CCTGTG |
| Sfi1 | ATCCGAGGACCTTCACC | GACAAGGCAAGCTGAA |
| | TAGAA | AGGAAG |
| Six1 | AGGTCATCGGAAGCTCT | GATACATCATTTTGCC |
| | TTTGT | CCAGGC |
| Socs2 | GTAGTCCTCCATCTCAA | TGCACCTGTATAGCGT |
| | GGCAG | GACATT |
| Piwil4 | GTCAGTGCTTGAGGTAA | CCTGAAGTTAAACCCC |
| | ATTCTCA | ACTATGT |
| Plzf | CTTCACTTGCCTCCAGT | TACACAGAAGGAAGGC |
| | CCAGA | AGGTGT |
| Ddx4 | CAGCTTCAGTAGCAGCA | CATGACTCGTCATCAA |
| | CAAG | CTGGA |
| Dazl | GATGGACATGAGATCAT | ATACCAGGGAGCAATC |
| | TGGAC | CTGAC |
| Piwil2 | GCTCGAAGACAACATTG | GGCCTTGGTCATAGAC |
| | TCCAG | TCCAAA |
| Itga6 | CACTGGCTTTAAAGGAC | TTGTTGTTGAACTCCC |
| | ACAGC | TCCCAA |
| Cd9 | GGATTGTTCTTCGGGTT | TCCTTGCTCCGTAACT |
| | CCTCT | TTTGGT |
| Kit | CAGTTACCGCGCTCTGT | GCCCCTTAAGTACCTG |
| | TTG | ACATCC |
| GfraI | TTTTACTGACAGTTGCG | TGAATGTGCTTCTGCT |
| | TCCAC | CAAAGTG |
| Ret | AGACTGCTGCTTTCACA | CACAGCACCACAGACT |
| | TCCTT | ATGTTC |
| Id4 | TCCCTTGCAGAGCTTTT | ACCAGAGAGCTGTTAC |
| | GCTAT | CTCTGA |
| Nanos2 | GACCTCATGGGACTGAT | CCAAGCCAACCTCCTA |
| | GACTG | GATAGC |

Bisulfite Ref: (Lee et al., 2009)

| | | |
|---|---|---|
| H19 | GGAATATTTGTGTTTTT | TTAAACCCCAACCTCT |
| | GGAGGG | ACTTTTATAAC |
| Meg3IG | GGTTTGGTATATATGGA | ATAAAACACCAAATCT |
| | TGTATTGTAATATAGG | ATACCAAAATATACC |
| Igf2r | TTAGTGGGGTATTTTTA | AAATATCCTAAAAATA |
| | TTTGTATGG | CAAACTACACAA |
| Peg10 | GTAAAGTGATTGGTTTT | TTAATTACTCTCCTAC |
| | GTATTTTTAAGTG | AACTTTCCAAATT |
| Snrpn | AATTTGTGTGATGTTTG | ATAAAATACACTTTCA |
| | TAATTATTTGG | CTACTAAAATCCACAA |

### Combined Bisulfite Restriction Analysis (COBRA)

COBRA was performed as previously reported (Lee et al., 2009). GSCs/GSCLCs were collected by FACS for AAG fluorescence, the genomic DNA thereof and tail genomic DNA of wild-type mouse were extracted, and 2 μg of DNA purified from each sample was subjected to bisulfite treatment, and purified using the Epitect Plus Bisulfite Kit (Qiagen) following the manufacturer's instructions. Using ExTaq (TakaRa), purified DNA was amplified by PCR by a protocol of 40 cycles at 96° C. for 30 sec, 60° C. for 1 min, and 72° C. for 1 min. The PCR primer sequences used are shown in Table 2. The amplified DNA was digested with appropriate restriction enzymes (New England BioLabs)-PhuI-HF (H19), HhaI (Peg10), TaqαI (Igf2r), AciI (Meg3 IG and Snrpn), and the digested samples were separated through electrophoresis in 2% or 3% agarose gel.

### (Results)

### PGCLC Undergoes Male Differentiation in Reconstituted Testes

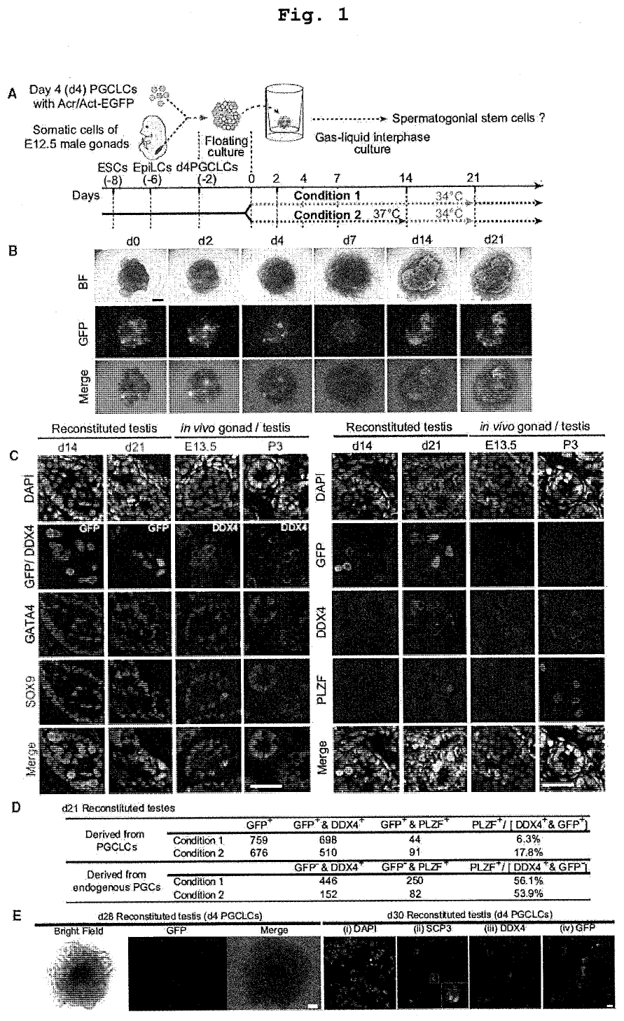

Close interactions between germ cells and testis somatic cells, particularly Sertoli cells, are essential for male germ cell differentiation (Svingen and Koopman, 2013). To investigate whether PGCLCs undergo differentiation for spermatogenesis in vitro, development of a culture system was tried using reconstituted testes (FIG. 1A). Embryonic stem cells (ESC) (129/SvJcl×C57BL/6 background) with Acro/Act-EGFP (AAG) transgene (Ohta et al., 2000) were induced into PGCLCs, and PGCLCs were isolated on day 4 (d4) or day 6 (d6) based on high levels of SSEA1 and INTEGRINβ3 using fluorescence-activated cell sorting (FACS). Aggregates of PGCLCs and embryonic testis cells at embryonic day (E)12.5 depleted of PGCs by magnetic cell separation (MACS) were created (FIG. 1A). Reconstituted testes were placed on a permeable membrane for gas/liquid interfacial culture (Steinberger et al, 1964) under either conditions for culturing for 2 days under floating conditions and then culturing at 34° C. (Condition 1) or conditions for culturing for 2 weeks at 37° C. and then at 34° C. for the remaining period (Condition 2) (FIG. 1A). d4 PGCLCs were used as the starting material because d6 PGCLCs were not sufficiently incorporated into the reconstituted testes (data not shown) for unknown reasons. Since similar results were obtained under any of Conditions 1 and 2, representative results from either of the Conditions are shown.

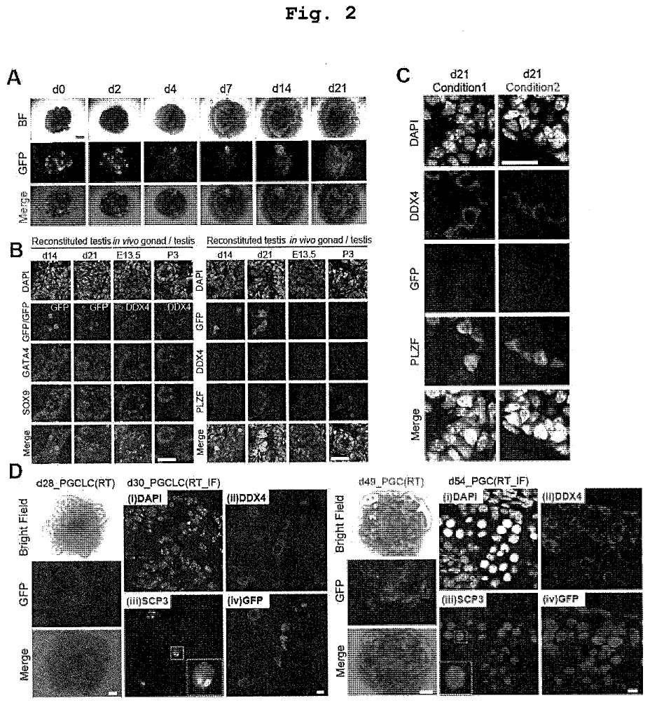

At d0 in the gas/liquid interfacial culture, the reconstituted testes had a flat, round shape with no distinct basic structure (FIGS. 1B and 2A). AAG-positive(+) cells showed a random distribution throughout the reconstituted testes with several aggregates formed (FIGS. 1B and 2A). From about d4, seminiferous tubule-like structures started to appear, a wide range of developments were shown and an anastomotic network was constructed at d7 (FIGS. 1B and 2A). By d7, AAG(+) cells, including aggregates outside of the seminiferous tubule-like structure, disappeared and most of the AAG(+) cells were inside the seminiferous tubule-like network (FIGS. 1B and 2A). Thereafter, with further development of the seminiferous tubule-like network, the reconstituted testes were stably maintained, and an increase in the number of AAG(+) cells in the network was observed (FIGS. 1B and 2A).

Immunofluorescence (IF) analysis of reconstituted testes at d14 and d21 clarified a robust seminiferous tubule-like structure described by cells positive for GATA4 and SOX9 (Vidal et al., 2001; Viger et al., 1998), which are important transcription factors of Sertoli cells. This was below the layer of squamous cells, most likely myoid cells (FIGS. 1C and 2B). AAG(+) cells with characteristic nuclear structure were almost exclusively present in the luminal compartment of tubule at d14, and many of them were found in the basal compartment at d21 (FIGS. 1C and 2B). At d14, DDX4 (Fujiwara et al., 1994), which is a germ cell marker that starts to be expressed by many AAG(+) cells of gonad PGCs, became positive(+), but PLZF(ZBTB16) (Buaas et al, 2004; Costoya et al, 2004), which is an SSC marker that begins to be expression inpro-spermatogonia at perinatal period was negative (FIGS. 1C and 2B). At d21, some of the AAG(+) cells showed (+) for both DDX4 and PLZF (FIGS. 1C and 2B). Under each Condition, sections of whole reconstituted testes at d21 were tested: the majority of AAG(+) cells became DDX4(+) ((˜92% in Condition 1, ˜75% in Condition 2) and only a portion of DDX4(+) cells became PLZF(+) ((˜6.3% under Condition 1, ˜18% under Condition 2) (FIG. 1D). Interestingly, endogenous PGCs that remained depleted by MACS (AAG(−)/DDX4(+)) acquired PLZF at a higher frequency than d4 PGCLCs (˜56% in Condition 1 and ˜54% in Condition 2) (FIGS. 1D and 2C).

A longer culture of the reconstituted testes were performed (up to d54). A small number of AAG(+) cells or endogenous germ cells were positive for SCP3 (Yuan et al., 2000), which is an important marker of meiosis initiation (FIGS. 1E and 2D); however, no cell was found that completed meiosis under the conditions of this experiment. In summary fashion, these data indicate that reconstituted testes reproduce testis development in vitro and that PGCLCs differentiate to form spermatogonium-like cells in reconstituted testes. The dynamics of differentiation of PGCLCs into such cell type was prolonged compared to that of PGCs in vivo.

In Vitro Propagation of Spermatogonium-Like State from PGCLC

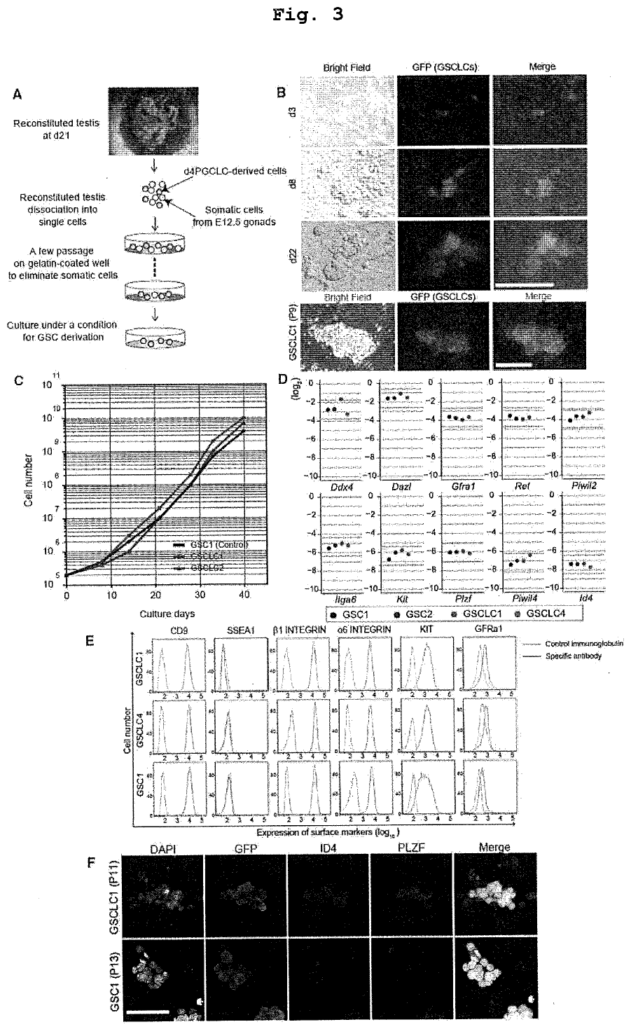

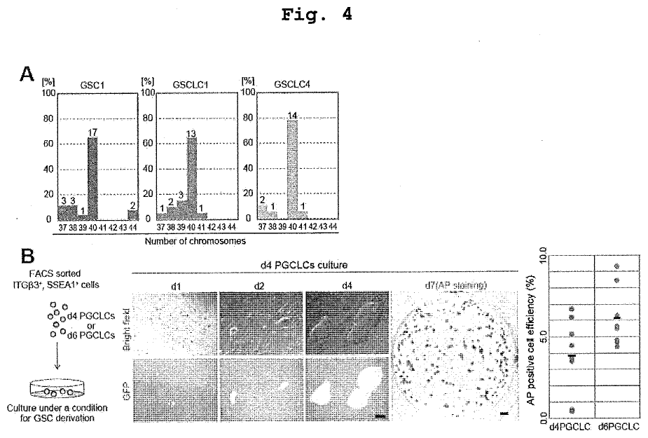

Perinatal pro-spermatogonia, spermatogonia, or SSCs, but not PGCs, can be propagated in vitro as a primary cell line with the capacity for self-renewal and spermatogenesis, referred to as germline stem cells (GSCs) (Kanatsu-Shinohara et al., 2003; Kubota et al., 2004). Neonatal testes are a robust source for the induction of GSC (Kanatsu-Shinohara et al., 2003). Therefore, it was tested whether GSC-like cells (GSCLCs) could be obtained from d21 reconstituted testes that had spermatogonium-like cells derived from PGCLCs and could resemble neonatal testes. The d21 reconstituted testes were dissociated into single cells, and AAG (+) cells were concentrated and cultured under GSC-induction conditions (FIG. 3A). At d3 of culture, AAG(+) cells were scattered on mouse embryonic feeders (MEFs) as single or paired cells (thousands of cells from single reconstituted testes), some of which formed small colonies at d8 (dozens of colonies at most) (FIG. 3B). Thereafter, such colonies showed slow proliferation and, after several passages, proliferated as stable cell lines with normal karyotype and a grapes-like colony form indistinguishable from GSC (9 and 6 strains respectively from Conditions 1 and 2), and had high efficiency for cryopreservation/re-expansion and subsequent thawing (FIGS. 3B and 4A). When the initial colony count was 10 or more, such cell lines could be established in a consistent manner and proliferated like GSCs (129/SvJcl×C57BL/6) (FIG. 3C) (Kanatsu-Shinohara) et al., 2003).

We compared the expression of key genes (Ddx4, Dazl, Gfra1, Ret, Piwil2, Itga6, Kit, Plzf, Piwil4, and Id4), surface markers(CD9, SSEA1, INTEGRINβ1, INTEGRINα6, KIT, and GFRα1), and transcription factors (PLZF and ID4) (Kanatsu-Shinohara and Shinohara, 2013; Yang and Oatley, 2014) in reconstituted testis-derived cell lines with those in GSCs, which revealed that they exhibit similar gene expression to GSCs (FIGS. 3D-3F). Therefore, these cell lines were named GSCLCs. To determine whether GSCLCs could be obtained from PGCLCs without formation of reconstituted testes, d4 or d6 PGCLCs sorted by SSEA1 and INTEGRINβ3 were directly cultured under GSC induction conditions. This, however, resulted in a rapid expansion (within a few days) of strongly alkaline phosphatase-positive, dome-shaped ESC-like colonies with efficiencies similar to those of embryonic germ cell (EGC) derivation from PGCs (˜5%) (FIG. 4B) (Matsui et al., 1992; Resnick et al., 1992), and we were unable to isolate/detect GSC-like colonies from such cultures. The present inventors concluded that the differentiation of PGCLCs into a male germline pathway is essential for the induction of GSCLCs.

Spermatogenesis in Adult Testes and GSCLC-Derived Fertile Offspring with Propagation Ability

Spermatogonia/SSCs can be colonized in adult testes (more than about 8 weeks) for spermatogenesis (Brinster and Zimmermann, 1994); however, PGCs colonized only in neonatal testes (˜5-10 days) and undergo spermatogenesis (Chuma et al., 2005; Ohta et al., 2004). This may be due to differences in either the homing ability or the inherent/acquired ability for spermatozoon between these cell types.

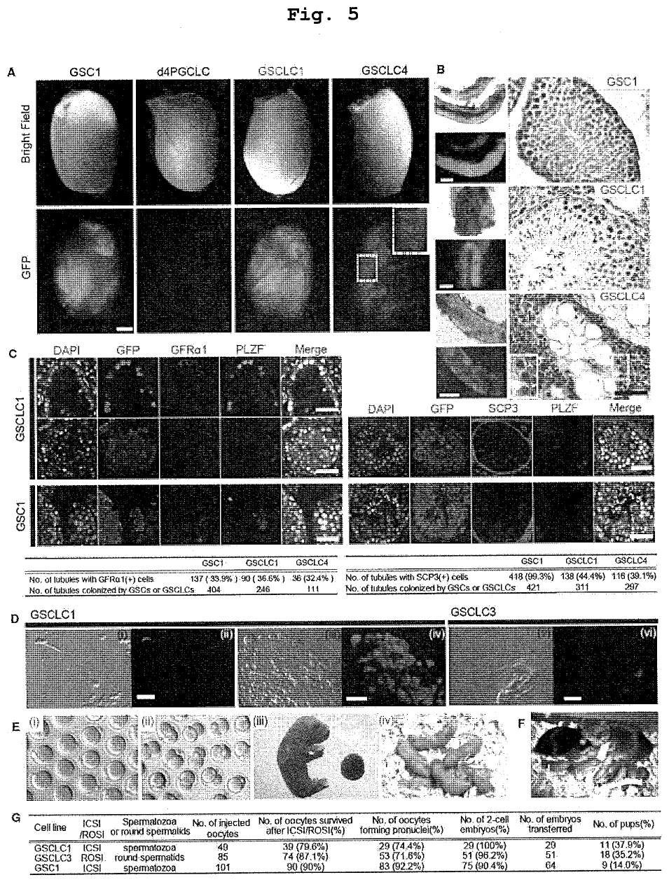

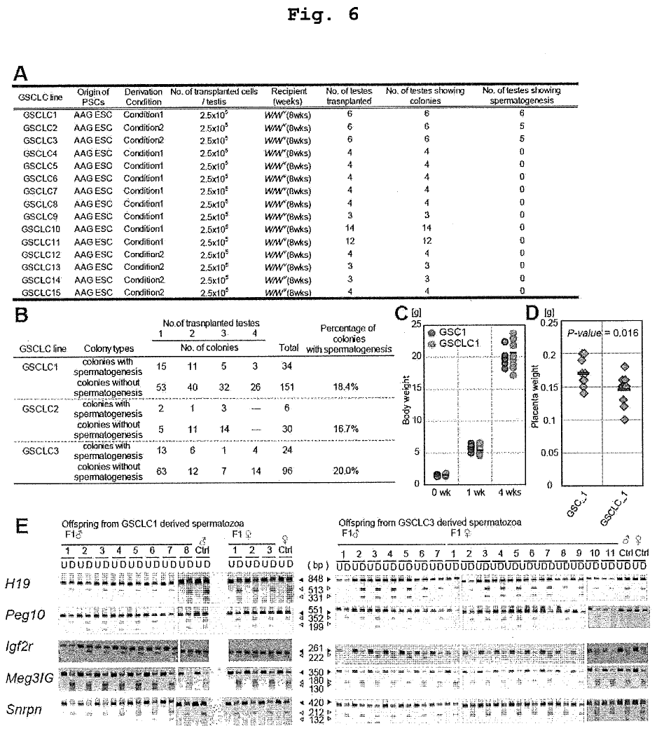

To examine whether GSCLCs acquire a mature stem cell property, we transplanted them into adult testes (8-10 weeks) of W/W^{v} mice. GSCs (AAG (+); 129/SvJcl×C57BL/6) (GSC1) robustly colonized adult testes and underwent spermatogenesis (FIG. 5A), whereas PGCLCs failed to show such activity in adults (FIG. 5A), although they did so in neonates (Hayashi et al., 2011). What is to be noted is, unlike PGCLCs, all GSCLC cell lines were colonized in adult testes (FIGS. 5A, 6A, and 6B). However, unexpectedly, a small number thereof (GSCLC 1, 2, 3: 3/15) underwent spermatogenesis in the fraction of the colonized tubule (FIGS. 5A, 6A, and 6B). Histological analysis confirmed that proper spermatogenesis occurred in the tubule completely occupied by AGS(+) cells derived from GSCLCs (FIG. 5B), but only spermatogonia or cells in the first meiosis phase were present in a tubule holding a chain of AAG(+) cells aligned only around the basal compartment (FIG. 5B). The GSCLC cell line did not form teratoma in the transplanted testes for at least 16 weeks post-transplantation.

IF analysis clarified that GFRα1(+) spermatogonia/SSC-like cells were present in similar proportions in tubule with GSC (˜33.9%) and GSCLC (GSCLC1: ˜36.6%; 4: ˜32.4%) colonized therein (FIG. 50). In contrast, almost all tubules with GSC colonization showed SCP3(+) meiocytes (˜99.3%), whereas they were less than half in tubules with GSCLC colonization (GSCLC1: 44.4%; 4:39.1%) (FIG. 5C). Therefore, while GSCLCs colonized in adult seminiferous tubule and exhibit spermatogonium/SSC characteristics, they tend to discontinue spermatogenesis in early stages of first meiosis or near the entry stage.

The function of spermatozoa or spermatids derived from GSCLCs was examined. Such cells (GSCLC1: spermatozoa; 3: round spermatid) could be consistently isolated from seminiferous tubules with successful spermatogenesis (FIG. 5D), intra-cytoplasmic sperm injection (ICSI) or round spermatid injection (ROSI) into the cytoplasm was performed, and apparently normal offspring was produced at a normal proportion (FIG. 5E and FIG. 5G). Such offspring was examined by Combined Bisulfite Restriction Analysis (COBRA) to find that it had an appropriate imprint, exhibited overall normal development, and were fertile (FIGS. 5F and 6C-6E). Thus, through PGCLC and differentiation thereof into the male pathway in reconstituted testes, PSCs can be induced into stable cell lines with the ability of SSCs which are an immediate precursor for spermatogenesis in adult testes.

### Example 2

### (Outline of Method)

Differentiation Induction from ES Cell into PGCLC and Culture of PGCLC

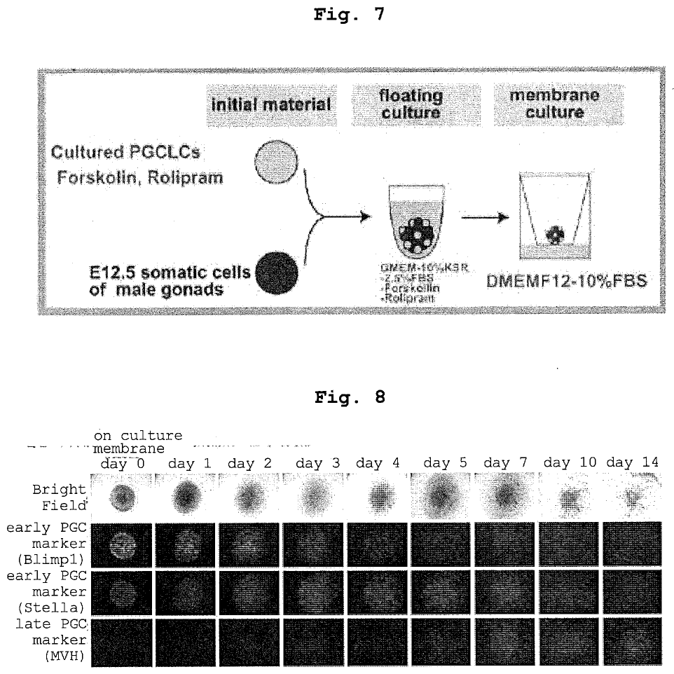

PGCLCs on day 4 (d4) induced in the same manner as in Example 1 were sorted by a cell sorter, and the population of cells positive for Blimp1, which is an initial PGC marker, was collected. The cells were seeded on feeder cells (m220 cell; Nature, 352: 809-811 (1991), J. Biol. Chem., 269: 1237-1242(1994)), and cultured for 5, 7, 9 days under medium conditions of GMEM-10% KSR-2.5% FBS-Forskolin-Rolipram-SCF addition (GK10FR) (FIG. 7).

### Reconstituted Testes of Cultured PGCLC and Male Fetal Gonad Cell

Cultured PGCLCs and male gonad somatic cells at gestational age of 12.5 days were mixed at a ratio of 5,000 cells:70,000 cells, and subjected to a floating culture for aggregation for 2 days in GK10FR (FIG. 7). Thereafter, the aggregates were transferred onto a culture insert, and gas/liquid interfacial culture was performed for 2 weeks (FIG. 7). The medium used was DMEM/F12-10% FBS.

Establishment of Spermatogonium-Like Cell Lines (GSCLCs) from Reconstituted Testes

GSCLCs were established from the reconstituted testes on day 10, day 14 from the start of the gas/liquid interfacial culture on the culture insert. The reconstituted testes were reacted in a 0.05% Trypsin solution at 37° C. for 10 min and dissociated into single cells by pipetting. Thereafter, the population of cells positive for DDX4(MVH), which is a late stage PGC marker was sorted by a cell sorter, seeded on a feeder cells (MEFs), and cultured in the same manner as in Example 1 under the conditions of StemPro34 as a basal medium and GDNF, FGF2, EGF, LIF addition.

### (Results)

Cultured PGCLCs Differentiated into Late Stage PGCs in Reconstituted Testes

The cultured PGCLCs proliferated until day 3 from the start of gas/liquid interfacial culture, and the tubular structure of the reconstituted testes became clear by day 4-5. During this period, the expression of the initial PGC marker Blimp1 and sequentially Stella was attenuated, and the expression of the late stage PGC marker MVH increased (FIG. 8). The expression of MVH increased toward day 10 of gas/liquid interfacial culture, and was thereafter slightly attenuated toward day 14.

GSCLCs can be Established from Reconstituted Testes on Day 10, Day 14

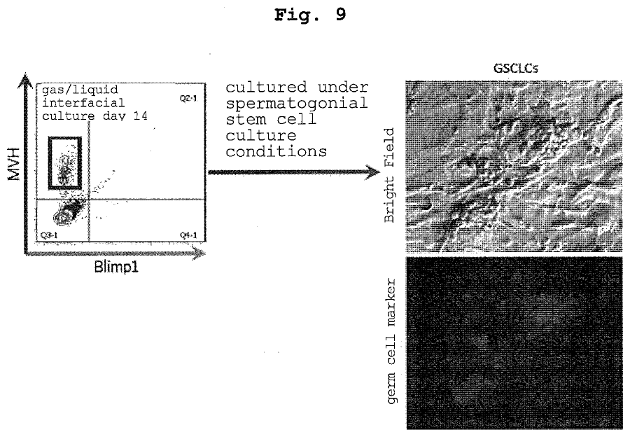

The reconstituted testes cultured for 10 days or 14 days were dissociated into single cells, and late stage PGC marker MVH-positive cells collected by a cell sorter were cultured under spermatogonial stem cell culture conditions. As a result, cell lines permitting long-term passage could be induced. A total of 4 lines (3 lines from reconstituted testes on day 10 and 1 line from reconstituted testes on day 14) could be established (FIG. 9).

### INDUSTRIAL APPLICABILITY

The germline stem cell-like cell (GSCLC) obtained by the method of the present invention permits long-term maintenance/expansion culture, can achieve fertile spermatogenesis when transplanted to not only neonate but also adult testes, and thus can greatly contribute to the promotion of the elucidation of the detailed development mechanism of germ cell and elucidation of the mechanism of the onset of sterility and hereditary diseases.

### INDICATION OF RELATED APPLICATION

This application is based on a patent application No. 2017-113054 filed in Japan (filing date: Jun. 7, 2017), the contents of which are hereby incorporated by reference in full herein.

1. A method for producing a spermatogenic stem cell-like cell from a primordial germ cell-like cell (PGCLC) derived from an isolated pluripotent stem cell (PSC) in vitro, the method comprising
(1) a step of coculturing PGCLC with a gonad somatic cell in suspension to give reconstituted testis, and
(2) a step of culturing the obtained reconstituted testis at gas/liquid interface to induce a DDX4-positive and PLZF-positive cell in the reconstituted testis.

2. A method for producing a GSC-like cell (GSCLC), comprising dissociating a spermatogenic stem cell-like cell obtained by the method according to claim 1 from the reconstituted testis, and culturing the cell under conditions that can induce a germline stem cell (GSC) from the spermatogenic stem cell.

3. An isolated GSCLC having the following properties:
(a) derived from isolated PSC,
(b) having expression levels equivalent to those of GSC as regards
(i) a gene selected from the group consisting of Ddx4, Daz1, Gfra1, Ret, Piwil2, Itga6, Kit, Plzf, Piwil4 and Id4,
(ii) a surface marker selected from the group consisting of CD9, SSEA1, INTEGRINβ1, INTEGRINα6, KIT and GFRα1, and
(iii) a transcription factor selected from the group consisting of PLZF and ID4,
(c) can be maintained or expanded at a proliferation rate equivalent to that of GSC,
(d) when GSCLC is transplanted into an adult testis,
(i) a proportion of seminiferous tubule having GFRα1-positive cells to seminiferous tubule with colonized transplanted cells is equivalent to that when GSC is transplanted, and
(ii) a proportion of seminiferous tubule having SCP3-positive cells to seminiferous tubule with colonized transplanted cells is lower than that when GSC is transplanted, and
(e) microinsemination with a sperm cell obtained by transplantation of the GSCLC into an adult testis produces normal offspring.

4. A method for producing a fertile sperm cell comprising transplanting GSCLC obtained by the method according to claim 2 into the testis of a mammal.

5. A method for producing an offspring with contribution of isolated PSC to the whole body, comprising fertilizing an oocyte by a sperm cell obtained by the method according to claim 4.

6. A method for producing a fertile sperm cell comprising transplanting GSCLC according to claim 3 into the testis of a mammal.

7. A method for producing an offspring with contribution of isolated PSC to the whole body, comprising fertilizing an oocyte by a sperm cell obtained by the method according to claim 6.
